(19) United States
(12) Patent Application Publication
(10) Pub. No.: US 2021/0299275 A1
(43) Pub. Date: Sep. 30, 2021

(54) GENE THERAPY FOR THE TREATMENT OF GALACTOSEMIA

(71) Applicant: RESEARCH INSTITUTE AT NATIONWIDE CHILDREN'S HOSPITAL, Columbus, OH (US)

(72) Inventor: Scott Allen Loiler, Columbus, OH (US)

(73) Assignee: RESEARCH INSTITUTE AT NATIONWIDE CHILDREN'S HOSPITAL, Columbus, OH (US)

(21) Appl. No.: 17/272,194

(22) PCT Filed: Aug. 30, 2019
(86) PCT No.: PCT/US2019/049157

§ 371 Date: Feb. 26, 2021

Related U.S. Application Data
(60) Provisional application No. 62/725,225, filed on Aug. 30, 2018.

(51) Int. Cl. A61K 48/00; C12N 9/12; C12N 15/86; C07K 14/005; A61P 3/00

(52) U.S. Cl. CPC A61K 48/005; C12Y 207/07012; C12N 9/1241; C12N 15/86; C12N 2750/14152; A61P 3/00; C12N 2750/14143; C12N 2750/14122; C07K 14/005

(57) ABSTRACT

Provided herein are recombinant AAV vectors encoding a GALT protein. Also provided in this disclosure are compositions and methods for the treatment of galactosemia.

### CROSS REFERENCE TO RELATED APPLICATION

This application claims priority under 35 U.S.C. § 119(e) to U.S. Provisional Ser. No. 62/725,225, Aug. 30, 2018, the contents of which are hereby incorporated by reference herein.

### BACKGROUND

The present disclosure relates generally to the field of gene therapy and in particular, for the treatment of Galactosemia.

Currently there are no treatment options for Galactosemia patients other than dietary restriction of galactose containing food. Even with strict adherence to a galactose free diet, many patients suffer from long-term mental and physical deficits due to small amounts of galactose found in foods and galactose produced endogenously. Thus a need exists in the art for an effective treatment. This disclosure satisfies this need and provides related advantages as well.

### SUMMARY

The disclosed gene therapy provides a sustained, long-term treatment option for Galactosemia patients by efficiently delivering the GALT gene to their cells, including the brain, to provide a sustained expression of functional GALT protein and alleviation of disease symptoms.

To achieve this therapy, provided herein is a polynucleotide or a recombinant adeno-associated viral (“AAV”) vector comprising, or alternatively consisting essentially of, or yet further consisting of a polynucleotide sequence encoding galactose-1-phosphate uridyl transferase (“GALT”). In one aspect, the polynucleotide sequence encoding the GALT comprises, or consists essentially of, or yet further consist of, a nucleotide sequence at least 85% identical to SEQ ID NO: 1. In one aspect, the sequence is at least 85% identical to SEQ ID NO: 1 with the provisio that at least one or more of the nucleotides that have been modified from the wild type sequence are not modified from SEQ ID NO: 1 (see FIG. 4). In another aspect, the polynucleotide sequence encoding GALT comprises, or consists essentially of, or yet further consist of, the nucleotide sequence set forth in SEQ ID NO: 1. In another embodiment, the polynucleotide sequence encodes an amino acid sequence of SEQ ID NO:4 or an equivalent thereof.

In one aspect, the vector is a self-complementary vector or a single stranded DNA (ssDNA) vector.

In one aspect, the recombinant adeno-associated viral (“AAV”) comprises, or consists essentially of, or yet further consists of, a AAV, such as a scAAV or a ssAAV flanked by two Inverted Terminal Repeats (ITRs). These ITRs form hairpins at the end of the sequence to serve as primers to initiate synthesis of the second strand before subsequent steps of infection can begin. The second strand synthesis is considered to be one of several blocks to efficient infection. Additional advantages of scAAV include increased and prolonged transgene expression in vitro and in vivo. Thus, in one aspect, the AAV further comprises two ITRs.

Non-limiting examples of recombinant AAV backbones to create the vector include AAV vector serotypes from the group of AAV1, AAV2, AAV4, AAV5, AAV6, AAV7, AAV8, AAV9, AAV10, AAV11, AAV12, AAV13, AAV PHP.B, or AAV rh74. In a further aspect, the vector backbone is an AAV9 serotype, an rh74 serotype, or a modified AAVrh74 serotype. Also provided is a polynucleotide encoding a modified AAVrh74 VP1 capsid protein comprising one or more modifications selected from the group of a substitution of isoleucine for asparagine at amino acid position 502, and an optional substitution of tryptophan to arginine at amino acid 505 of the VP1 of AAVrh74, and an insertion of the peptide YIG or YIGSR at amino acid position 591 of the VP1 of AAVrh74, or an equivalent thereof, or a polynucleotide that is at least 85% identical with the proviso that one or more, or two or more, or three or more, or four or more modifications are identical to the amino acids in the modifice AAVrh74 VP1 capsid protein and the complements of these polynucleotides. Also provided is modified AAVrh74 VP1 capsid protein comprising one or more modifications selected from the group of a substitution of isoleucinefor asparagine at amino acid position 502, and an optional substation of tryptophan to arginine at amino acid 505 of the VP1 of AAVrh74, and an insertion of the peptide YIG at amino acid position 591 of the VP1 of AAVrh74, or an equivalent thereof, or a polynucleotide that is at least 85% identical with the proviso that one or more, or two or more, or three or more, or four or modifications are identical to the amino acids in the modifice AAVrh74 VP1 capsid protein.

The polynucleotide sequence encoding GALT is optionally operably linked to a promoter, a tissue-specific control element, or a constitutive promoter. Non-limiting examples of constitutive promoters include, for example, a Rous sarcoma virus (RSV) LTR promoter (optionally with the RSV enhancer), a cytomegalovirus (CMV) promoter, an SV40 promoter, a dihydrofolate reductase promoter, a j-actin promoter, a phosphoglycerol kinase (PGK) promoter, or an EF1 promoter. In a particular aspect, the j-actin promoter is a chicken β-actin (“CBA”) promoter. In another embodiment, the promoter is selected from a CMV promoter, an EF1a promoter, an SV40 promoter, a PGK1 (human or mouse) promoter, a P5 promoter, a Ubc promoter, a human beta actin promoter, a CAG promoter, a TRE promoter, a UAS promoter, an Ac5 promoter, a polyhedrin promoter, a CaMKIIa promoter, a Gall promoter, a TEF1, a GDS promoter, an ADH1 promoter, a CaMV35S promoter, a Ubi promoter, an H1 promoter, a U6 promoter, or an Alpha-1-antitrypsin promoter.

In a further aspect, the polynucleotide or recombinant AAV further comprises a polynucleotide encoding an enhancer element. Non-limiting examples include a CMV enhancer, a WPRE and a RSV enhancer.

In a particular aspect, the recombinant polynucleotide or AAV vector comprises, or alternatively consists essentially of, or yet further consists of a nucleotide sequence at least 85% identical to SEQ ID NO: 2 or SEQ ID NO: 3. In one aspect, the sequence is at least 85% identical to SEQ ID NO: 2 or 3 with the provisio that at least one or more of the nucleotides that have been modified from the wild type GALT (see FIG. 4) sequence are not modified from SEQ ID NO: 2 or 3. In a further aspect, the recombinant AAV vector, comprises, or alternatively consists essentially of, or yet further consists nucleotide sequence set forth in SEQ ID NO: 2 or SEQ ID NO: 3.

The recombinant polynucleotide or AAV vector as described herein can further comprise a detectable or purification marker. The polynucleotides or vectors can be contained as compositions comprising the polynucleotides or the vectors and a carrier, such as a preservative or pharmaceutically acceptable carrier.

Also provided is a cell or a viral particle which comprises the polynucleotides and/or AAV vectors as described herein. The cells can be prokaryotic or eukaryotic and can include packaging cell lines. The cells and viral particles can be contained as compositions comprising the vectors and a carrier, such as a preservative or pharmaceutically acceptable carrier.

The polynucleotides and vectors are useful in therapeutic or other methods and are contained within wildtype or modified capsid proteins. Thus, provided herein are AAV viral particles comprising a recombinant polynucleotide and/or AAV vector as disclosed herein, as well as compositions containing the polynucleotide and/or viral particles for use in the methods as disclosed herein. Further provided are methods to prepare vectors, polynucleotides and viral particles containing them. In one aspect, a method is provided for introducing a functional GALT enzyme into a cell, comprising contacting the cell with a recombinant polynucleotide and/or AAV vector or a capsid or viral particle containing the same, or a composition as described herein. The contacting can be ex vivo or in vivo.

Also provided is a method for treating galactosemia in a subject in need thereof, comprising or alternatively consisting essentially of, or yet further consisting of administering to the subject an effective amount of the recombinant polynucleotide and/or AAV vector, the viral particle comprising the recombinant polynucleotide and/or AAV vector, or a composition as described herein.

Also provided is a method of increasing galactose metabolism in a subject that may, in one aspect, be suffering from galactosemia the method comprising or alternatively consisting essentially of, or yet further consisting of administering to the subject the recombinant polynucleotide and/or AAV vector, the viral particle, or a composition as described herein. In one aspect, the amount delivered is an effective amount as determined by the treating professional.

Yet further provided is a method of reducing a disease condition or symptom associated with galactosemia in a subject suffering from galactosemia comprising, or alternatively consisting essentially of, or yet further consisting of administering to the subject the recombinant polynucleotide and/or AAV vector, the viral particle, or a composition as described herein, wherein the disease condition comprises one or more of jaundice, hepatosplenomegaly, hepatocellular insufficiency, hypoglycemia, renal tubular dysfunction, muscle hypotonia, sepsis, cataract, ataxia, tremor, decreased bone density, fertility in adult females, impaired motor functions and growth restriction in the GalT-deficient, or primary ovarian insufficiency. In one aspect, the method further comprises identifying a subject as suffering from galactosemia, and then treating the subject identified by the method. In one aspect, the amount delivered is an effective amount as determined by the treating professional.

In each of these methods, the galactosemia to be treated is a type 1 galactosemia, a type 2 galactosemia, or a type 3 galactosemia. In a particular aspect, the galactosemia is the type 1 galactosemia.

The recombinant polynucleotide and/or AAV vector, the viral particle, or a composition is administered by intramuscular injection or intravenous injection. Alternatively, the recombinant polynucleotide and/or AAV vector, the polynucleotide and/or viral particle, or a composition is administered systemically. Yet further, the recombinant polynucleotide and/or AAV vector, the viral particle, or a composition is parentally administration by injection, infusion or implantation. In a further aspect, the polynucleotide, the recombinant AAV vector, the viral particle, or a composition is administered by intramuscular injection or intravenous injection and then subsequently systemically.

A kit is also provided by this disclosure, the kit comprising the recombinant polynucleotide and/or AAV vector, the viral particle, and/or the composition as described herein. The kits can optionally contain instructions for making and using the polynucleotides, vectors, viral particles or compositions.

### BRIEF DESCRIPTION OF THE DRAWINGS

FIGS. 1A-1C show the maps of three exemplary vectors: pAAV-CB-GALT-WPRE-kan (FIG. 1A), pAAV-CB-hGALT (FIG. 1B), and pAAV-CB-hGALT-WPREv2 (FIG. 1C).

FIG. 2 shows bio-distribution of the pAAV-CB-GALT-WPRE-kan vector (FIG. 1A).

FIG. 3 shows expression of human GALT protein expressed using the vector of the pAAV-CB-GALT-WPRE-Kan vector (FIG. 1A).

FIG. 4 is a sequence alignment of the codon optimized recombinant polynucleotide encoding GALT as described herein as compared to corresponding wild-type sequence.

FIG. 5 shows AAV9 vector bio-distribution. C57Bl/6J (WT) or GalT^{-/-} (CG) female mice were injected intravenously with 1E+12 viral genomes (Vg) of AAV9/lucEYFP reporter virus and 1 week later tissues were harvested and assayed for vector genome copies by qPCR.

FIG. 6 shows protein expression. Plasmid pAAV-CB-GALT-WPRE or pAAV-CB-lucEYFP reporter plasmid was transfected into HEK293 cells and two days later assayed for GALT expression by Western blot analysis. GALT transfected cell lysate (30 or 3 μg), LucEYFP cell lysate (138 μg), HepG2 endogenous GALT protein control (135 μg), purified bacterially expressed GALT protein standard with slightly higher molecular weight due to purification tag.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this invention pertains.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the invention and the appended claims, the singular forms “a,” “an” and “the” are intended to include the plural forms as well, unless the context clearly indicates otherwise.

As used herein, the term “comprising” is intended to mean that the compositions and methods include the recited elements, but do not exclude others. As used herein, the transitional phrase consisting essentially of (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74.

The term “galactosemia” refers to a genetic disorder that affects a subject's ability to metabolize the galactose. Lactose in food (such as dairy products) is broken down by the enzyme lactase into glucose and galactose. In individuals with galactosemia, the enzymes needed for further metabolism of galactose (Galactokinase and galactose-1-phosphate uridyltransferase) are severely diminished or missing entirely, leading to toxic levels of galactose or galactose 1-phosphate (depending of which enzyme is missing) in various tissues as in the case of classic galactosemia, resulting in diseases conditions, which include but are not limited to, jaundice, hepatomegaly (an enlarged liver), renal tubular dysfunction, muscle hypotonia, sepsis, hepatocellular insufficiency, cirrhosis, renal failure, cataracts, vomiting, seizure, hypoglycemia, lethargy, brain damage, and ovarian failure. Thus, these conditions can also be suitably treated by the methods of this disclosure when related to galactosemia in the subject.

Based on the enzyme that is deficient in the subject, galactosemia can be categorized as type 1 galactosemia (galactose-1-phosphate uridyl transferase), type 2 galactosemia (galactokinase), and type 3 galactosemia (UDP galactose epimerase). In one embodiment, the recombinant vectors or the methods as disclosed herein can be used to treat type 1, type 2, or type 3 galactosemia. In another embodiment, the recombinant or the methods can be used to treat type 1 galactosemia. Because galactosemia is associated with a number of disease conditions, in another embodiment, the disclosure provides a method of reducing a disease condition in a subject suffering from galactosemia, wherein the disease condition is selected from jaundice, hepatomegaly (an enlarged liver), renal tubular dysfunction, muscle hypotonia, sepsis, hepatocellular insufficiency, cirrhosis, renal failure, cataracts, vomiting, seizure, hypoglycemia, lethargy, brain damage, or ovarian failure.

Other disease conditions associated with galactosemia include but are not limited to speech deficits, Ataxia, Friedreich's Ataxia, dysmetria, diminished bone density, or premature ovarian failure. These conditions also can be treated by the methods of this disclosure.

The term “cell” as used herein may refer to either a prokaryotic or eukaryotic cell, optionally obtained from a subject or a commercially available source.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells and 293T cells.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene” refers to a polynucleotide containing at least one open reading frame (ORF) that is capable of encoding a particular polypeptide or protein after being transcribed and translated. A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell. In one aspect, this invention provides promoters operatively linked to the downstream sequences, e.g., suicide gene, VEGF, 165A VEGF, tet activator, etc.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a 3-actin promoter, a phosphoglycerol kinase (PGK) promoter, a U6 promoter, or an EF1 promoter. In some embodiments, the promoter is a chicken 3-actin (“CBA”) promoter (e.g., base pairs numbered 439 to 708 of SEQ ID NO: 2 or base pairs numbered 644 to 921 of SEQ ID NO: 3, or an equivalent of each thereof).

Additional non-limiting exemplary promoters with certain target specificity are provided herein below including but not limited to CMV, EF1a, SV40, PGK1 (human or mouse), P5, Ubc, human beta actin, CAG, TRE, UAS, Ac5, Polyhedrin, CaMKIIa, Gall, TEF1, GDS, ADH1, CaMV35S, Ubi, H1, U6, and Alpha-1-antitrypsin promoter. Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some embodiments, the promoter is coupled to an enhancer to increase the transcription efficiency. Non-limiting examples of enhancers include an RSV enhancer or a CMV enhancer (e.g., base pairs numbered 153 to 432 of SEQ ID NO: 2).

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In another embodiment, the promoter is an inducible promoter. In a specific related embodiment, the promoter an inducible tetracycline promoter. The Tet-Off and Tet-On Gene Expression Systems give researchers ready access to the regulated, high-level gene expression systems described by Gossen & Bujard (1992; Tet-Off) and Gossen et al. (1995; Tet-On). In the Tet-Off system, gene expression is turned on when tetracycline (Tc) or doxycycline (Dox; a Tc derivative) is removed from the culture medium. In contrast, expression is turned on in the Tet-On system by the addition of Dox. Both systems permit gene expression to be tightly regulated in response to varying concentrations of Tc or Dox. Maximal expression levels in Tet systems are very high and compare favorably with the maximal levels obtainable from strong, constitutive mammalian promoters such as CMV (Yin et al., 1996). Unlike other inducible mammalian expression systems, gene regulation in the Tet Systems is highly specific, so interpretation of results is not complicated by pleiotropic effects or nonspecific induction. In E. coli, the Tet repressor protein (TetR) negatively regulates the genes of the tetracycline-resistance operon on the Tn10 transposon. TetR blocks transcription of these genes by binding to the tet operator sequences (tetO) in the absence of Tc. TetR and tetO provide the basis of regulation and induction for use in mammalian experimental systems. In the Tet-On system, the regulatory protein is based on a “reverse” Tet repressor (rTetR) which was created by four amino acid changes in TetR (Hillen & Berens, 1994; Gossen et al., 1995). The resulting protein, rtTA (reverse tTA also referred to tetracycline activator protein), is encoded by the pTet-On regulator plasmid. This gene may be in a separate vector as the GALT gene or encoded on the same gene.

In a related embodiment, the vector further comprises, or alternatively consists essentially of, or yet further consists of a nucleic acid encoding a tetracycline activator protein; and a promoter that regulates expression of the tetracycline activator protein.

Other inducible systems useful in vectors, isolated cells, viral packaging systems, and methods described herein include regulation by ecdysone, by estrogen, progesterone, chemical inducers of dimerization, and isopropyl-beta-D1-thiogalactopyranoside (EPTG).

As used herein, the term “recombinant expression system” or “recombinant vector” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. Ads do not require integration into the host cell genome. Recombinant Ad derived vectors, particularly those that reduce the potential for recombination and generation of wild-type virus, have also been constructed. Such vectors are commercially available from sources such as Takara Bio USA (Mountain View, Calif.), Vector Biolabs (Philadelphia, Pa.), and Creative Biogene (Shirley, N.Y.). Wild-type AAV has high infectivity and specificity integrating into the host cell's genome. See, Wold and Toth (2013) Curr. Gene. Ther. 13(6):421-433, Hermonat & Muzyczka (1984) Proc. Natl. Acad. Sci. USA 81:6466-6470, and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In some embodiments, the signal peptide is removed following localization. Examples of signal peptides are well known in the art. Non-limiting examples are those described in U.S. Pat. Nos. 8,853,381, 5,958,736, and 8,795,965.

As used herein, the term “viral capsid” or “capsid” refers to the proteinaceous shell or coat of a viral particle. Capsids function to encapsidate, protect, transport, and release into host cell a viral genome. Capsids are generally comprised of oligomeric structural subunits of protein (“capsid proteins”). As used herein, the term “encapsidated” means enclosed within a viral capsid.

As used herein, the term “helper” in reference to a virus or plasmid refers to a virus or plasmid used to provide the additional components necessary for replication and packaging of a viral particle or recombinant viral particle, such as the modified AAV disclosed herein. The components encoded by a helper virus may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

In some embodiments, the AAV is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. U.S. Pat. No. 9,434,928 also provide the seequences of the capsid proteins and a self-complementary genome. In one aspect, the genome is a self-complementary genome. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise a nucleic acid molecule encoding GALT (e.g., SEQ ID NO: 1) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV PHP.B and AAV rh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

As used herein, the term “exterior” in reference to a viral capsid protein refers to the surface, domain, region, or terminal end of the capsid protein that is exterior-facing in an assembled viral capsid. The term “interior” in reference to a viral capsid protein refers to the surface, domain, region, or terminal end (amino-terminus end or carboxy terminus) of the capsid protein that is interior-facing in an assembled viral capsid. When used in reference to an assembled viral capsid, the term “interior” refers to the encapsidated space inside the viral capsid and the inward-facing surface of the capsid that is exposed to the enclosed space. The interior space is encapsidated by viral capsid proteins and may comprise nucleic acids such as the viral genome, viral proteins, proteins of the host or packaging cell, and any other components or factors packaged or encapsidated during replication, virion assembly, encapsidation, and/or packaging.

As used herein, the term “conjugated” refers to any method of attaching, coupling, fusing, and/or linking a viral capsid protein to a GALT protein or an equivalent thereof. Non-limiting examples of conjugation include recombinant fusion proteins wherein the GALT protein or an equivalent thereof and the viral capsid protein are encoded by a single polynucleotide that comprises the genes for both the GALT protein or an equivalent thereof and the viral capsid protein, modular intein based assembly of a GALT-intein protein and a viral capsid-intein protein, posttranslational modification that causes a chemical bond to form between a GALT protein or equivalent thereof and the viral capsid protein, and linkage of a GALT or equivalent thereof and a viral capsid protein via one or more linkers. In some embodiments, conjugation may be a temporary or transient state of association between the viral capsid protein and the equivalent thereof. For example, the GALT or an equivalent thereof may be transiently linked to the viral capsid protein via a polymer sensitive to a change in pH or ion gradient at a later step in infection or within a particular cell microenvironment, such as oxime linkage (see, e.g. Jin et al. Biomacromolecules, 2011, 12 (10), pp 3460-3468 and Yoshida et al. Expert Opin Drug Deliv. 2013 November; 10(11): 1497-1513).

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

A “composition” is intended to mean a combination of active polypeptide, polynucleotide or antibody and another compound or composition, inert (e.g., a detectable label) or active (e.g., a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject” of diagnosis or treatment is a cell or an animal such as a mammal, or a human. A subject is not limited to a specific species and includes non-human animals subject to diagnosis or treatment and are those subject to infections or animal models, for example, simians, murines, such as, rats, mice, chinchilla, canine, such as dogs, leporids, such as rabbits, livestock, sport animals, and pets. Human patients are included within the term as well.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, in some embodiments the effective amount is the amount sufficient to result in regaining part or full function of a gene that is deficient in a subject. In other embodiments, the effective amount of an AAV viral particle is the amount sufficient to result in expression of a gene in a subject. In some embodiments, the effective amount is the amount required to increase galactose metabolism in a subject in need thereof. The skilled artisan will be able to determine appropriate amounts depending on these and other factors.

In some embodiments the effective amount will depend on the size and nature of the application in question. It will also depend on the nature and sensitivity of the target subject and the methods in use. The skilled artisan will be able to determine the effective amount based on these and other considerations. The effective amount may comprise one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating vetrenarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

### Modes for Carrying Out the Disclosure

### AAV Vectors, Capsids and Methods of Preparation

Provided herein is a recombinant polynucleotide or adeno-associated viral (“AAV”) vector comprising, or alternatively consisting essentially of, or yet further consisting of, the polynucleotide sequence that encodes galactose-1-phosphate uridyl transferase (“GALT”). In one aspect, the polynucleotide sequence encoding the GALT comprises, or consists essentially of, or yet further consist of, a nucleotide sequence at least 85%, or 90%, or 95%, or 97%, or 99% identical to SEQ ID NO: 1. In one aspect, the sequence is at least 85%, or 90%, or 95%, or 97%, or 99% identical to SEQ ID NO: 1 with the provisio that at least one, or two, or three, or four, or five, or six, or seven, or eight, or nine, or ten or more, or all of the nucleotides that have been modified from the wild type sequence are not modified from SEQ ID NO: 1. In another aspect, the polynucleotide sequence encoding GALT comprises, or consists essentially of, or yet further consist of, the nucleotide sequence set forth in SEQ ID NO: 1. In another embodiment, the polynucleotide sequence encodes an amino acid sequence of SEQ ID NO:4 or an equivalent thereof.

In one aspect, the vector is a self-complementary vector or a single stranded DNA (ssDNA) vector.

In one aspect, the AAV comprises, or consists essentially of, or yet furher consists of, a AAV, such as a scAAV or a ssAAV flanked by two Inverted Terminal Repeats (ITRs). These ITRs form hairpins at the end of the sequence to serve as primers to initiate synthesis of the second strand before subsequent steps of infection can begin. The second strand synthesis is considered to be one of several blocks to efficient infection. Additional advantages of scAAV include increased and prolonged transgene expression in vitro and in vivo. Thus, in one aspect, the AAV further comprises two ITRs.

Non-limiting examples of recombinant AAV backbones to create the vector include AAV vector serotypes from the group of AAV1, AAV2, AAV4, AAV5, AAV6, AAV7, AAV8, AAV9, AAV10, AAV11, AAV12, AAV13, AAV PHP.B, or AAV rh74. In a further aspect, the vector backbone is an AAV9 serotype, an rh74 serotype, or a modified AAVrh74 serotype.

The polynucleotide sequence encoding GALT is optionally operably linked to a promoter, a tissue-specific control element, or a constitutive promoter. Non-limiting examples of constitutive promoters include, for example, a Rous sarcoma virus (RSV) LTR promoter (optionally with the RSV enhancer), a cytomegalovirus (CMV) promoter, an SV40 promoter, a dihydrofolate reductase promoter, a j-actin promoter, a phosphoglycerol kinase (PGK) promoter, or an EF1 promoter. In a particular aspect, the j-actin promoter is a chicken β-actin (“CBA”) promoter. In another embodiment, the promoter is selected from a CMV promoter, an EF1a promoter, an SV40 promoter, a PGK1 (human or mouse) promoter, a P5 promoter, a Ubc promoter, a human beta actin promoter, a CAG promoter, a TRE promoter, a UAS promoter, an Ac5 promoter, a polyhedrin promoter, a CaMKIIa promoter, a Gall promoter, a TEF1, a GDS promoter, an ADH1 promoter, a CaMV35S promoter, a Ubi promoter, an H1 promoter, a U6 promoter, or an Alpha-1-antitrypsin promoter.

In a further aspect, the recombinant polynucleotide or AAV further comprises a polynucleotide encoding an enhancer element. Non-limiting examples include a CMV enhancer, WPRE and a RSV enhancer.

In a particular aspect, the recombinant polynucleotide or AAV vector, comprises, or alternatively consists essentially of, or yet further consists of a nucleotide sequence at least 85%, or 90%, or 95%, or 97%, or 99% identical to SEQ ID NO: 2 or SEQ ID NO: 3. In one aspect, the sequence is at least 85%, or 90%, or 95%, or 97%, or 99% identical to SEQ ID NO: 2 or 3 with the provisio that at least one or two, or three, or four, or five, or six, or seven, or eight, or nine, or ten or more, or all of the nucleotides that have been modified from the wild type sequence are not modified from SEQ ID NO: 2 or 3. In a further aspect, the recombinant AAV vector, comprises, or alternatively consists essentially of, or yet further consists nucleotide sequence set forth in SEQ ID NO: 2 or SEQ ID NO: 3.

The recombinant polynucleotide or AAV vector as described herein can further comprise a detectable or purification marker. The polynucleotides and/or vectors can be contained as compositions comprising the vectors and a carrier, such as a preservative or pharmaceutically acceptable carrier.

AAV vector delivery currently relies on the use of serotype selection for tissue targeting based on the natural tropism of the virus or by the direct injection into target tissues. If systemic delivery is required to achieve maximal therapeutic benefit, then serotype selection is the only available option for tissue targeting combined with tissue specific promoters. Thus, the AAV vectors can be packaged into capsid with tissue tropism.

In some aspects, the vector is encapsulated in a viral capsid that is a wild-type or a modified capsid particle. In one aspect, the disclosure provides capsid proteins, isolated polynucleotides, methods for the preparation of capsid proteins, recombinant viral particles and recombinant expression systems for the generation of viral particles. In one embodiment, the viral capsid protein that comprises, or alternatively consists essentially of, or yet further consists of, a wild-type capsid protein or alternatively, a viral capsid protein modified by amino acid substitution or insertion of between 1 to 7 amino acid. In some embodiments, the viral capsid protein is a VP1, optionally of AAV9, AAV PHP.B, or a modified AAVrh74. For example, the AAV PHP.B has a modified amino acid 498 of AAV9 VP1 from asparagine to lysine to reduce the liver tropism. In further embodiments, the modification comprises the substitution of isoleucine for asparagine at amino acid position 502 of the VP1 of AAVrh74 or an equivalent modification. In some embodiments, the modification comprises the substation of tryptophan to arginine at amino acid 505 of the VP1 of AAVrh74. In some embodiments, the modification is an insertion of the peptide YIG or YIGSR at amino acid position 591 of the VP1 of AAVrh74. In some embodiments, this peptide has a high affinity for Alpha 7 beta 1 integrin and/or is positioned in a region that is likely to alter normal rh74 receptor binding. Also provided are equivalents of these polypeptides and polynucleotides encoding them, wherein the equivalent has at least 85%, or 90%, or 95%, or 97, or 99% sequence identity with the provisio that one or more, two or more, three or more, four or more, five or more, six or more, seven or more, or all of the amino acid and polynucleotides are not altered from the mutated sequences.

The virus, e.g., AAV, can be packaged using a viral packaging system such as a retroviral, adenoviral, herpes virus, or baculovirus packaging system. In some embodiments, packaging is achieved by using a helper virus or helper plasmid and a cell line. The helper virus or helper plasmid contains elements and sequences that facilitate the delivery of genetic materials into cells. In another aspect, the helper plasmid or a polynucleotide comprising the helper plasmid is stably incorporated into the genome of a packaging cell line, such that the packaging cell line does not require additional transfection with a helper plasmid.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV. The viral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The virus is a leukemia virus such as a Moloney Murine Leukemia Virus (MMLV), the Human Immunodeficiency Virus (HIV), or the Gibbon Ape Leukemia virus (GALV).

The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The helper plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the helper plasmids and the plasmids encoding the AAV viral proteins are transiently cotransfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the invention this transiently transfected first population of cells is then cocultivated with mammalian target cells to transduce the target cells with the foreign gene at high efficiencies.

### Packaging Systems

The invention also provides a viral packaging system comprising: the vector as described above, wherein the backbone is derived from a plasmid, a virus; a packaging plasmid; and an envelope plasmid. The packaging plasmid contains the nucleoside, capsid and matrix proteins. Examples of packaging plasmids are also described in the patent literature, e.g., U.S. Pat. Nos. 7,262,049; 6,995,258; 7,252,991 and 5,710,037, incorporated herein by reference. The system also contains a plasmid encoding a pseudotyped envelope protein provided by an envelope plasmid. Pseudotyped viral vectors consist of vector particles bearing glycoproteins derived from other enveloped viruses or alternatively containing functional portions. See, for example U.S. Pat. No. 7,262,049, incorporated herein by reference. In a preferred aspect, the envelope plasmid encodes an envelope protein that does not cause the viral particle to unspecifically bind to a cell or population of cells. The specificity of the viral particle is conferred by the antibody binding domain that is inserted into the particle. Examples of suitable envelope proteins include, but are not limited to those containing the Staph. aureus ZZ domain. The choice of glycoprotein for use in the envelope is determined in part, by the antibody to which the particle may be conjugated.

This disclosure also provides the suitable packaging cell line. In one aspect, the packaging cell line is the HEK-293 cell line. Other suitable cell lines are known in the art, for example, described in the patent literature within U.S. Pat. Nos. 7,070,994; 6,995,919; 6,475,786; 6,372,502; 6,365,150 and 5,591,624, each incorporated herein by reference.

This invention further provides a method for producing an pseudotyped AAV particle, comprising, or alternatively consisting essentially of, or yet further consisting of, transducing a packaging cell line with the viral system as described above, under conditions suitable to package the viral vector. Such conditions are known in the art and briefly described herein. The pseudotyped viral particle can be isolated from the cell supernatant, using methods known to those of skill in the art, e.g., centrifugation. Such isolated particles are further provided by this invention.

This invention further provides the isolated polynucleotides and/or AAV viral particles produced by this method. The pseudotyped viral particle comprises, or alternatively consists essentially of, or yet further consists of a polynucleotide as described herein and encoding a GALT protein or an equivalent thereof (e.g., SEQ ID NO. 4 or an equivalent of SEQ ID NO. 4 as described above).

The isolated pseudotyped particles can be conjugate to one or more of an antibody or an antibody fragment (e.g. a fragment containing at least the Fc domain) that retains the ability to bind a pre-selected cell receptor.

The antibodies are not species specific. In other words, the antibodies can be polyclonal or monoclonal and can be murine, ovine, human or other species. In addition, they can be chimeric or humanized.

### Host Cells

Yet further provided is an isolated cell or population of cells, comprising, or alternatively consisting essentially of, or yet further consisting of, isolated polynucleotides, viral particles, vectors and packaging systems as described above and incorporated herein by reference. In one aspect, the isolated cell is a packaging cell line.

Also provided is an isolated cell or population of cells, comprising, or alternatively consisting essentially of, or yet further consisting of, a polynucleotide sequence encoding a GALT protein or an equivalent thereof as described herein and a constitutive or an inducible promoter that regulates expression of the nucleic acid encoding the GALT. In one embodiment, the promoter is an inducible promoter as described herein. In another aspect, the promoter is a constitutive promoter as described herein. In a further aspect, the nucleic acid encoding GALT comprises, or alternatively consists essentially of, or yet further consists of SEQ ID NO.: 1, or an equivalent thereof. In one aspect, the sequence is at least 85%, or 90%, or 95%, or 97%, or 99% identical to SEQ ID NO: 1 with the provisio that at least one, or two, or three, or four, or five, or six, or seven, or eight, or nine or ten or more, or all of the nucleotides that have been modified from the wild type sequence are not modified from SEQ ID NO: 1. In a further embodiment, the isolated cell further comprises, or alternatively consists essentially of, or yet further consists of a nucleic acid encoding a tetracycline activator protein; and a promoter that regulates expression of the tetracycline activator protein. In one embodiment, the promoter that regulates expression of the tetracycline activator protein is a constitutive promoter. In a related embodiment, the promoter is a phosphoglycerate kinase promoter (PGK) or a CMV promoter.

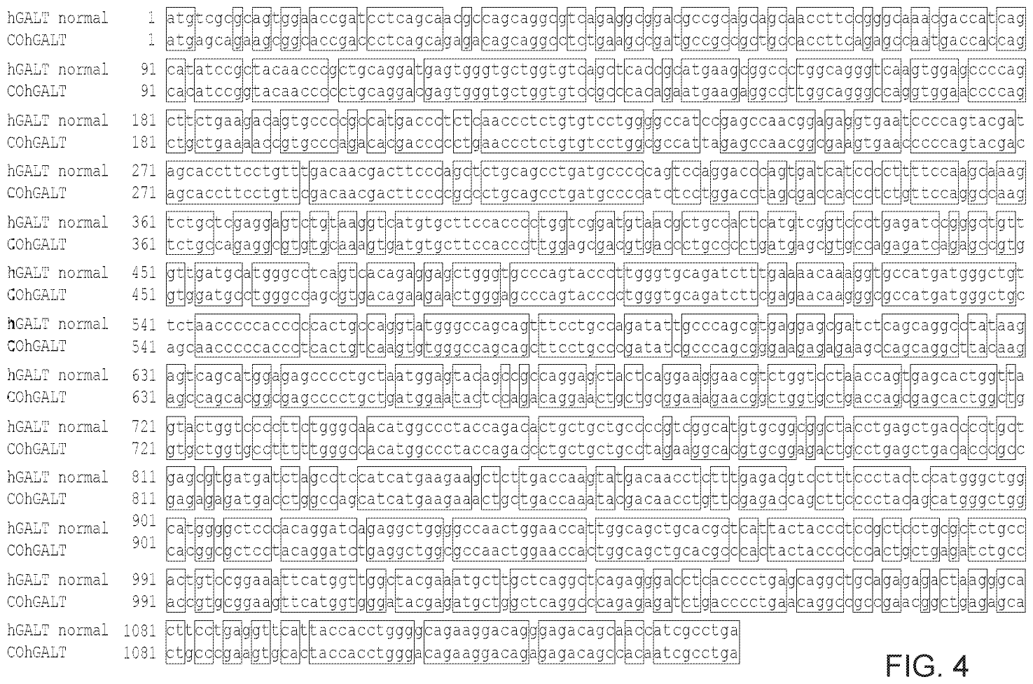

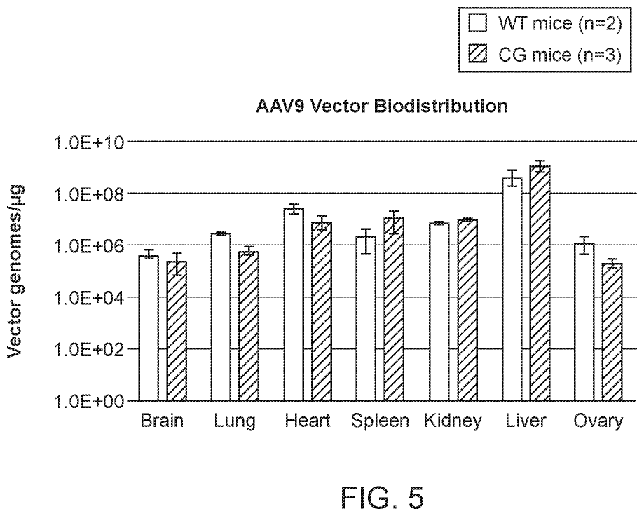

In a specific embodiment, the isolated cell comprises, or alternatively consists essentially of, or yet further consists of a nucleic acid comprising the polynucleotide of SEQ ID NO: 1 that encodes a GALT protein, or a biological equivalent thereof. In a related embodiment, the biological equivalent of GALT comprises a nucleic acid that hybridizes under conditions of high stringency to the complement of SEQ ID NO: 1 and encodes a GALT protein (e.g., SEQ ID NO 4). In another embodiment, the biological equivalent thereof comprises a nucleic acid having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to SEQ ID NO.: 1 and in one aspect, wherein at least one or two, or three, or four, or five, or six, or seven, or eight, or nine, or ten or more, or all of the nucleotides that have been modified from the wild type sequence are not modified from SEQ ID NO: 1 (see FIG. 4). In a further aspect, the GALT protein is a wild-type human GALT protein.

The isolated cells described herein can be any of a cell of a species of the group of: murine, rats, rabbit, simians, bovines, ovine, porcine, canines, feline, farm animals, sport animals, pets, equine, and primate, and in particular a human cell.

In certain embodiments, the isolated cell as described herein comprises a certain level of the GALT protein. The level of GALT protein can be achieved by selecting an appropriate constitutive promoter that produces the desirable level of protein or by using an inducible system that regulates the amount of protein produced. These promoters and inducible systems have previously been described. In one embodiment, the isolated cell comprises, or alternatively consists essentially of, or yet further, consists of at least about 1×10-7 ng, about 3×10-7 ng, about 5×10-7 ng, about 7×10-7 ng, about 9×10-7 ng, about 1×10-6 ng, about 2×10-6 ng, about 3×10-6 ng, about 4×10-6 ng, about 6×10-6 ng, about 7×10-6 ng, about 8×10-6 ng, about 9×10-6 ng, about 10×10-6 ng, about 12×10-6 ng, about 14×10-6 ng, about 16×10-6 ng, about 18×10-6 ng, about 20×10-6 ng, about 25×10-6 ng, about 30×10-6 ng, about 35×10-6 ng, about 40×10-6 ng, about 45×10-6 ng, about 50×10-6 ng, about 55×10-6 ng, about 60×10-6 ng, about 65×10-6 ng, about 70×10-6 ng, about 75×10-6 ng, about 80×10-6 ng, about 85×10-6 ng, about 90×10-6 ng, about 95×10-6 ng, about 10×10-5 ng, about 20×10-5 ng, about 30×10-5 ng, about 40×10-5 ng, about 50×10-5 ng, about 60×10-5 ng, about 70×10-5 ng, about 80×10-5 ng, or about 90×10-5 ng of GALT protein.

### Therapeutic Compositions and Methods

This disclosure provides a therapeutic gene vector compositions and therapies to treat galactosemia. Galactosemia is an orphan disease, which is cause by a recessive single gene defect in the GALT gene (Galactose-1-Phosphate Uridyltransferase). See ghr.nlm.nih.gov/gene/GALT. It affects approximately 1 in 45,000 newborns in the US. Fortunately, it is detected early with a nationwide newborn screening program. The only treatment is to remove all sources lactose and galactose from the child's diet. Early treatment with galactose restriction is beneficial and life-saving in the severe forms of the disease but usually there exists long-term complications such as language and cognitive impairment as well as physical impairments due to neuromuscular to neuromuscular and ovarian toxicities from low levels of galactose contained in other foods and endogenously produced galactose.

Various embodiments of the vector are described herein. In one embodiment, the vector provided herein and method using the vector comprises, or consists essentially thereof, or yet consisting of, administration of an effective amount of recombinant adeno-associated viral (“AAV”) vector comprising, or alternatively consisting essentially of, or yet further consisting of, a polynucleotide sequence encoding galactose-1-phosphate uridyl transferase (“GALT”) is provided by this disclosure. In one aspect, the polynucleotide sequence encoding the GALT comprises a nucleotide sequence at least 85%, or 90%, or 95%, or 97%, or 99% identical to SEQ ID NO: 1. In one aspect, the sequence is at least 85%, or 90%, or 95%, or 97%, or 99% identical to SEQ ID NO: 1 with the provisio that at least one or two, or three, or four, or five, or six, or seven, or eight, or nine, or ten or more, or all of the nucleotides that have been modified from the wild type sequence are not modified from SEQ ID NO: 1 (see FIG. 4). In another aspect, the polynucleotide sequence encoding GALT comprises the nucleotide sequence set forth in SEQ ID NO: 1. In another embodiment, the polynucleotide sequence encodes an amino acid sequence of SEQ ID NO:4. In one aspect, the polynucleotide and/or AAV vector contains a reporter gene either luciferase or GFP to determine biodistribution of the vector. In another embodiment, the polynucleotide and/or AAV vector comprises, or alternatively consists essentially of, or yet further consists of a polynucleotide sequence that comprises a GALT gene sequence from human (e.g., NCBI Reference Sequence: NM_000155.3, or NM_001258332.1), mouse (e.g., NCBI Reference Sequence: NM_001302511.1, or NM_016658.3), rat (e.g., NCBI Reference Sequence: NM_001013089.2), chimpanzee (e.g., NCBI Reference Sequence: XM_003951414.4 or XM_001163419.6), or other species. In another embodiment, the polynucleotide and/or AAV vector comprises, or alternatively consists essentially of, or yet further consists of a polynucleotide sequence that comprises a codon-optimized version of human GALT gene (SEQ ID NO. 1 and FIG. 4). One of the AAV vectors is identified as pscAAV-CB-hGALT (SEQ ID NO. 2). In another embodiment, the AAV vector is identified as pAAV-CB-hGALT-WPREv2 (SEQ ID NO. 3). The pscAAV-CB-hGALT vector comprises a constitutive CBA promoter with a small SV40 intron driving a codon-optimized version of human GALT gene (Galactose-1-Phosphate Uridyltransferase) (SEQ ID NO. 1 and FIG. 4).

In one aspect, the AAV vector comprises a polynucleotide sequence that encodes encodes a human GALT protein (e.g., SEQ ID NO. 4), a mouse GALT protein (e.g., NCBI Reference Sequence NP_001289440.1), a rat GALT protein (e.g., NCBI Reference Sequence: NP_001013107.1), a chimpanzee GALT protein (e.g., NCBI Reference Sequence: XP_003951463.1), or a GALT protein from other species. In another embodiment, the AAV vector comprises a polynucleotide sequence that encodes a human GALT protein (e.g., SEQ ID NO. 4).

In one aspect, AAV is an AAV9 serotype. Alternative serotypes or modified capsid viruses can be used to optimize neuronal tropism. Alternative vectors include: a modified AAV9 serotype vector for higher neuronal tropism than standard AAV9, e.g., PHP.B that uses a Cre-lox recombination system to identify neuronally targeted vectors. Alternatively, the AAV9 PHP.B has a modified amino acid 498 of VP1 from Asparagine to Lysine to reduce the liver tropism. Further variants of AAVrh74 that have mutated several amino acids can be used for very broad tissue tropism including the brain.

In one aspect the AAV vector is contained within a modified viral capsid protein that comprises, or alternatively consists essentially of, or yet further consists of, a viral capsid protein modified by amino acid substitution or insertion of between 1 to 7 amino acid. Applicant has generated three mutants. One of the mutants (AAVmut4, asparagine to isoleucine at amino acid 502 of VP1 capsid) increases gene delivery globally to all tissues tested up to 56-fold (between 3 and 56-fold increase depending on tissue) higher transduction efficiency. Another mutant (AAVmut5, tryptophan to arginine at amino acid 505 of VP1 capsid) increases gene delivery to the heart almost 50-fold over AAVrh74. A third mutant (AAVYIG591) targets a receptor found primarily on satellite cells which are considered muscle stem cells although the satellite cell tropism. Notably, based on Applicant's knowledge of AAV crystal structures and alpha 7 beta 1 integrin to design AAVYIG591 is believed to have a higher affinity for skeletal muscle and lower affinity for liver.

Not to be bound by theory, Applicant expects therapeutic benefits to the patient will be achieved by increasing the effective dose that reaches the muscle without increasing overall dose to the patient. By reducing the overall dose required to achieve a therapeutic benefit, fewer viral antigens are delivered to the patient, ideally resulting in reduced immune responses to the vector and increased safety. Manufacturing enough gene therapy drug product to conduct late stage clinical trials is a major hurdle in further development. Reducing the dose requirements to achieve therapeutic benefit will result in reduced manufacturing requirements, reduced costs of manufacturing, faster clinical trial development and greater ability to treat more patients.

Accordingly, this disclosure relates to AAV vectors contained within modified capsid proteins, isolated polynucleotides, methods for the preparation of modified capsid proteins, recombinant viral particles and recombinant expression systems for the generation of modified viral particles. One aspect of the disclosure relates to a modified viral capsid protein that comprises, or alternatively consists essentially of, or yet further consists of, a viral capsid protein modified by amino acid substitution or insertion of between 1 to 7 amino acid. In some embodiments, viral capsid protein is a VP1, optionally of AAV9, AAV PHP.B, or AAVrh74. In further embodiments, the modification comprises the substitution of isoleucine for asparagine at amino acid position 502 of the VP1 of AAVrh74 or an equivalent modification. In some embodiments, the modification comprises the substation of tryptophan to arginine at amino acid 505 of the VP1 of AAVrh74. In some embodiments, the modification targets a receptor found primarily on satellite cells, optionally muscle stem cells. In some embodiments, the modification is an insertion of the peptide YIG at amino acid position 591 of the VP1 of AAVrh74. In some embodiments, this peptide has a high affinity for Alpha 7 beta 1 integrin and/or is positioned in a region that is likely to alter normal rh74 receptor binding.

The plasmid backbones of two exemplary vectors are disclosed herein. The first vector contains a CBA promoter/enhancer-driving expression of a reporter fusion protein composed of luciferase and enhanced yellow fluorescent protein. In one aspect, the luciferase and enhanced yellow fluorescent protein sequences are deleted. This vector can be packaged as a single stranded virus inside of a standard AAV9 serotype capsid or a mutant capsid. In another aspect, the AAV contains a self-complementary vector with a CBA promoter/enhancer driving expression of a codon optimized human GALT gene (SEQ ID NO 1) or an equivalent thereof. This vector can be packaged in a standard AAV9 serotype capsid. This vector can provide high levels of human GALT protein. The luc-EYFP reporter virus can be evaluated for biodistribution and gene expression either by in-vivo luciferase staining at various time points using a Xenogen IVIS (or similar) or by harvesting tissues at various time points and assaying for gene expression of either EYFP or luciferase activity. Samples may additionally be obtained and evaluated vector genome quantification by qPCR. Administration can begin newborn-affected animals (day 1 or 2 after birth). In an animal model, mouse pups can be injected via the facial vein (see jove.com/video/52037/intravenous-injections-in-neonatal-mice) and look for the biodistribution of vector by GFP expression after 4 weeks. Homozygous GALT defective mice born from mothers fed a normal chow diet can provide a good indication of the vector tropism and biodistribution in the knock-out (KO) mouse model.

This disclosure also provides compositions comprising a carrier and one or more of a modified protein, a polynucleotide, vector, plasmid, host cell, or expression system. Further provided is a kit comprising one or more of a modified protein, a polynucleotide, vector, plasmid, host cell, or expression system and instructions for use. The compositions can be formulated for the specified mode of administration.

### Administration

Administration of the recombinant polynucleotide, vector (e.g., AAV), viral particle or compositions of this disclosure can be effected in one dose, continuously or intermittently throughout the course of treatment. Administration may be through any suitable mode of administration, including but not limited to: localized, intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation. In one embodiment, the recombinant vector or the composition is administered by intramuscular injection or intravenous injection. In another embodiment, the recombinant AAV vector or the composition is administered systemically. In another embodiment, the recombinant AAV vector or the composition is parentally administration by injection, infusion or implantation. In one aspect, the AAV vector or GALT polynucleotide is locally delivered to the liver.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 1E+9 vector genomes to as much as 1E+17 vector genomes per administration.

In some embodiments of the methods described herein, the number of viral particles (e.g., AAV) administered to the subject ranges administered to the subject ranges from about 10⁹ to about 10¹⁷. In particular embodiments, about 10¹⁰ to about 10¹², about 10¹¹ to about 10¹³, about 10¹¹ to about 10¹², about 10¹¹ to about 10¹⁴, about 5×10¹¹ to about 5×10¹², or about 10¹² to about 10¹³ viral particles are administered to the subject.

In a further aspect, the polynucleotide, viral particle and compositions of the disclosure can be administered in combination with other treatments, e.g., those approved treatments suitable for galactosemia and its associated disorders or conditions. A non-limiting example includes the treatment of galactosemia with the viral vectors or compositions of this disclosure, while reducing or eliminating lactose and/or galactose from a subject's diet.

Successful treatment and/or repair is determined when one or more of the following is detected: alleviation or amelioration of one or more of symptoms of the treated subject's disease, disorder, or condition, diminishment of extent of the subject's disease, disorder, or condition, stabilized (i.e., not worsening) state of a disease, disorder, or condition, delay or slowing of the progression of the disease, disorder, or condition, and amelioration or palliation of the disease, disorder, or condition. In some embodiments, success of treatment is determined by detecting the presence repaired target polynucleotide in one or more cells, tissues, or organs isolated from the subject. In some embodiments, success of treatment is determined by detecting the presence polypeptide encoded by the repaired target polynucleotide in one or more cells, tissues, or organs isolated from the subject.

In one embodiment, the recombinant polynucleotide and/or viral vector can repair the GALT gene in a subject. In some embodiments, the ratio of repaired target polynucleotide or polypeptide to unrepaired target polynucleotide or polypeptide in a successfully treated cell, tissue, organ or subject is about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to Western blot, Northern blot, Southern blot, PCR, sequencing, mass spectrometry, flow cytometry, immunohistochemistry, immunofluorescence, fluorescence in situ hybridization, next generation sequencing, immunoblot, and ELISA.

### Kits

The polynucleotides, agents, vectors, or compositions described herein may, in some embodiments, be assembled into pharmaceutical or diagnostic or research kits to facilitate their use in therapeutic, diagnostic or research applications. In some embodiments, the kits of the present disclosure include one or more of: modified viral capsid proteins, isolated polynucleotides, vectors, host cells, recombinant viral particles, recombinant expression systems, modified AAV, modified cells, isolated tissues, compositions, or pharmaceutical compositions as described herein.

In some embodiments, a kit further comprises instructions for use. Specifically, such kits may include one or more agents described herein, along with instructions describing the intended application and the proper use of these agents. As an example, in one embodiment, the kit may include instructions for mixing one or more components of the kit and/or isolating and mixing a sample and applying to a subject. In certain embodiments, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some embodiments, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some embodiments, the preservation solution contains an amount of metalloprotease inhibitors.

As used herein, “instructions” can define a component of instruction and/or promotion, and typically involve written instructions on or associated with packaging of the claimed methods, recombinant vectors, or compositions. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. In some embodiments, the written instructions are in a form prescribed by a governmental agency regulating the manufacture, use or sale of pharmaceuticals or biological products, which instructions can also reflect approval by the agency of manufacture, use or sale for animal administration.

In some embodiments, the kit contains any one or more of the components described herein in one or more containers. Thus, in some embodiments, the kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

The therapies as described herein can be combined with appropriate diagnostic techniques to identify and select patients for the therapy. For example, a genetic test to identify a mutation in a GALT gene can be provided. Thus, patients harboring a mutation can be identified as suitable for therapy.

### EXAMPLES

### In Vitro Administration

The viral particles are made by standard triple transfection method using plasmids for the vector genome, the AAV rep and cap genes and an Ad helper plasmid which provides the necessary Adenovirus helper functions to product AAV. These three plasmids are transfected into serum-free suspension grown HEK293 cells using PEI. Four days after transfection the viruses are harvested and purified by standard methods. The efficacious doses range in the 1012 to 1014 Vg/kg if given intravenously based on similar disease models and serotypes. The AAVmut4 comprises modifications to regions involved in liver specific binding which give the virus a broader tropism to many other target tissues including CNS and muscle.

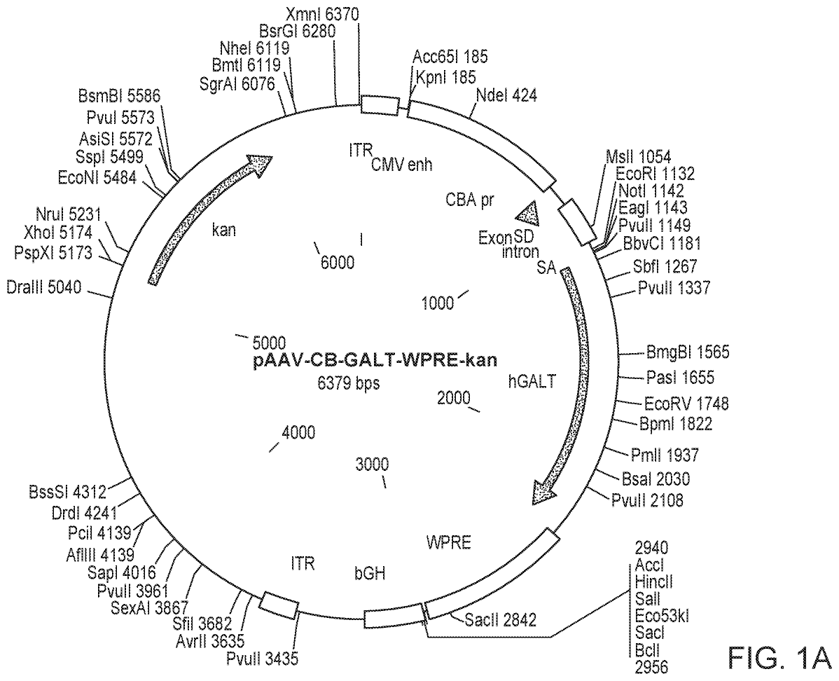

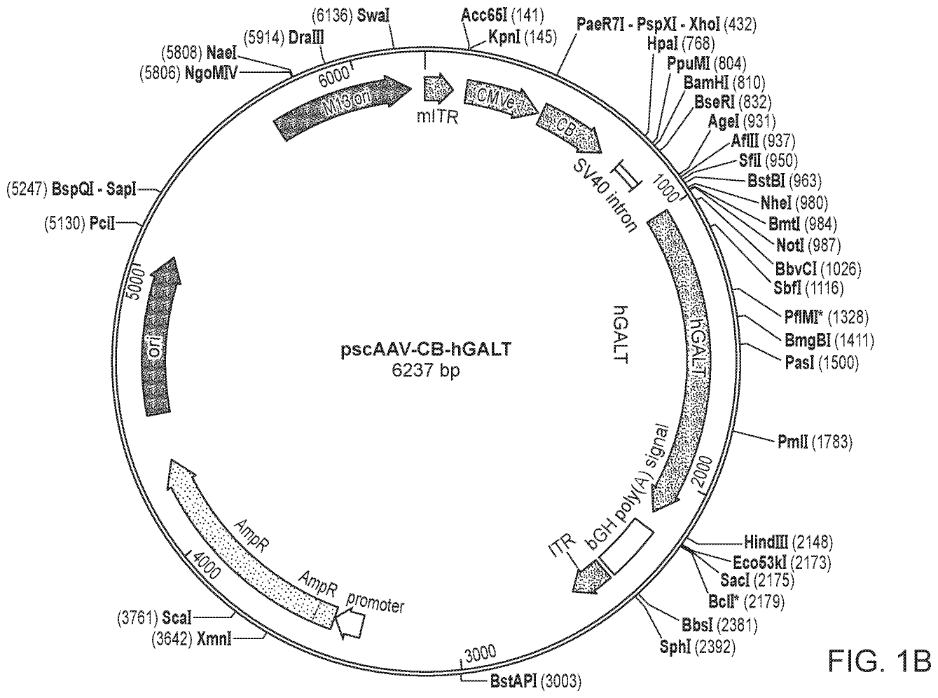

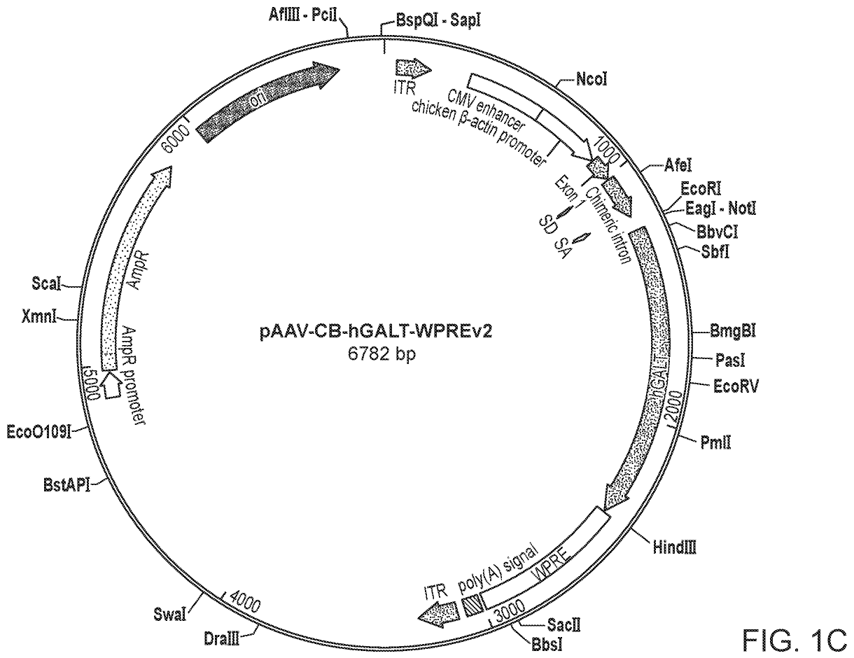

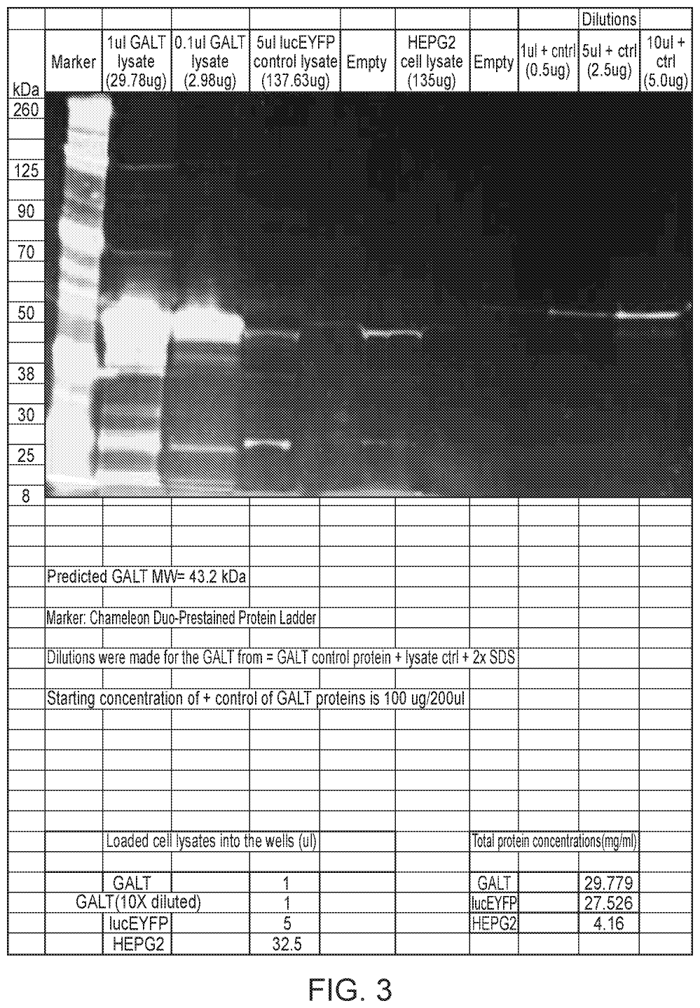

The plasmid pAAV-CB-GALT-WPRE-kan (FIG. 1) was transfected into HEK293 cells by transient transfection and two days later the cells were lysed and harvested proteins were run on an acrylamide gel, transferred to nylon membrane and probed with an anti-human GALT antibody followed by an IR dye labeled secondary antibody for detection. As shown in FIG. 3, lane 1 is a molecular weight marker with sizes indicated on Y axis. Lane 2 is 1 μl of GALT transfected cell lysate, lane 3 is 0.1 μl of GALT transfected cell lysate, lane 4 is 5 μl of lysate of HEK293 cells transfected with a lucEYFP reporter plasmid used as a transfection and cell control which also shows a low amount of native human GALT protein endogenously expressed, lane 5 is empty, lane 6 is a HepG2 cell lysate showing native human GALT protein, lane 7 is empty, lanes 8-10 show increasing concentrations of a bacterially expressed GALT protein standard with slightly higher mobility due to the tag used for purification.

### In Vivo Administration

The GALT gene therapy approach as disclosed herein is highly innovative because first, there is no AAV-mediated gene-based therapy or any gene-based therapy approved for treating the long-term complications associated with Classic Galactosemia. Second, in addition to the technology, the disclosed therapy can use localized gene delivery to the liver to normalize the aberrant galactose metabolism in distant organs.

A new GALT gene-trapped (GalT-deficient) mouse model was used as described in Tang, M. et al. Eur. J. Hum. Genet, (2014):1172-1179. Using a LC-MS/MS procedure (Li, Y. et al., Mol. Genet. Metab. (2011) 102(1):33-40, the total absence of GALT activity was confirmed in the homozygous GalT gene-trapped mice. Further characterization of the homozygous GalT gene-trapped mice revealed galactose sensitivity in the newborn GalT-deficient pups, reduced fertility in adult females, impaired motor functions and growth restriction in the GalT-deficient mice of both sexes (Tang, et al. (2014), supra; Balakrishnan, B. et al. (2016) 470(1):205-212; Chen et al. (2017) J. Inherit. Metab. Dis. 40(1):131-137).

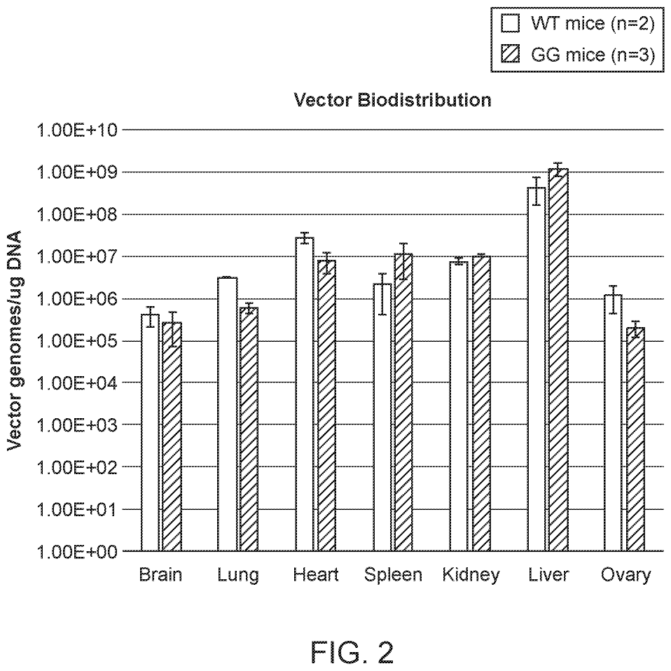

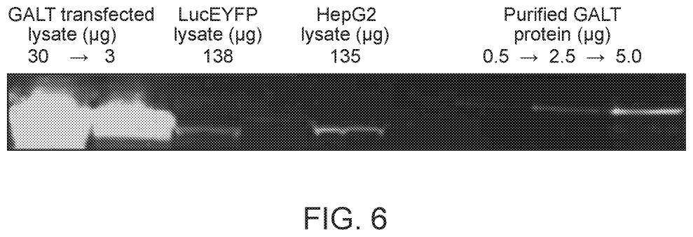

As shown in FIG. 6, transduction of AAV9-GALT vectors (see FIGS. 1 and 2) to HEK293 cell lines led to production of abundant GALT protein. GALT protein expression can be modified by substitution of the strong CBA promoter/enhancer with a promoter that will produce GALT protein close to the physiological levels as necessary.

Bio-Distribution: It has been established that AAV9 vector has broad bio-distribution, which include the central nervous system (CNS), while AAV8 primarily targets the liver in mice. Thus, administration of GALT and codon optimized GALT can be with an AAV8 vector. Delivery of the vector through the vasculature of 4-week-old wild-type and GalT-deficient mice can assess for any differences in bio-distribution between the two groups. As an initial matter, 1 E+12 Vg of each AAV vector expressing the lucEYFP reporter construct via tail vein into male and female mice (N=5 per sex per group, N based on FDA's guidelines for toxicity/bio-distribution studies as laid out in the document entitled “Guidance for Industry: Gene Therapy Clinical Trials—Observing Subjects for Delayed Adverse Events”) is done. Mice are imaged weekly for 4 weeks before sacrifice. Controls are age- and sex-matched animals injected with vectors without the GALT gene insert. After 4 weeks, animals are sacrificed and perfused with fixative, and brain, heart, kidney, liver, eyes and gonads are isolated. Tissues are sectioned to stain for EYFP microscopically, while a portion of each tissue will be used for qPCR determination of vector genomes per microgram of genomic DNA. In a another controlled administration, mice are challenged with 10% and 20% galactose in their diets.

Dosing Studies: Separately, male and female 4-week-old GalT-deficient mice are IV injected with one of three different doses of AAV9-GALT (or AAV8-GALT) vector at 1E+10, 1E+11 or 1E+12 vg, respectively per mouse, 12 mice per group (N=6 per sex per group), plus empty vector-injected control group of selected serotype. Animals are monitored for up to 6 months and examined for potential improvement in motor coordination and reproductive fitness. Animals are also monitored and assayed for any overt adverse physiological effects of the treatment, such as weight loss or inactivity.

Behavioral Analysis: Mice (n=12 per group) are tested for behavioral performance at 6 months of age, to quantify the functional impact of the treatment on the neurological disorders. The behavioral tests include cognitive and swimming ability in the Morris water maze, and motor function on a rotarod. To determine the ataxia-related motor impairment of mice, a modified rotarod to encourage the animal to walk along the rod alternating between limbs, instead of passively using its body for support will be used. The speed is gradually increased to 6 RPM over two minutes for training purposes. After resting for two-minutes, the mice are tested at 6 RPM for a maximum of two minutes (lower time if the animal fails the test). The trial can be repeated at 12 RPM for each mouse for increased challenge comparison to wild-type (WT) normal mice. Each mouse is tested three times at each speed after a five-minute resting period. At the end of 6 months, all mice are euthanized and evaluated for GALT gene expression by Western and vector genome content by qPCR in various regions of the brain and other tissues along with H&E histology by board certified pathologists for pathology.

Fertility assessment in female mice: Reproductive fitness of the female mice is assessed by monitoring their estrus cycle at regular time intervals and performing follicle count at the end of six months. Any signs of normalization of estrus cycle and follicle count alone are sufficiently good indicators for improved fertility in these animals.

### EQUIVALENTS

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

All technical and patent publications referenced herein are incorporated by reference.

### REFERENCES

1. Fang, H., et al., Comparison of adeno-associated virus serotypes and delivery methods for cardiac gene transfer. Hum Gene Ther Methods, 2012. 23(4): p. 234-41.
2. Bianconi, E., et al., An estimation of the number of cells in the human body. Ann Hum Biol, 2013. 40(6): p. 463-71.
3. Tran, T., et al., Laminin drives survival signals to promote a contractile smooth muscle phenotype and airway hyperreactivity. FASEB J, 2013. 27(10): p. 3991-4003.
4. Mori, S., et al., Biodistribution of a low dose of intravenously administered AAV-2, 10, and 11 vectors to cynomolgus monkeys. Jpn J Infect Dis, 2006. 59(5): p. 285-93.
5. Grimm, D., et al., In vitro and in vivo gene therapy vector evolution via multispecies interbreeding and retargeting of adeno-associated viruses. J Virol, 2008. 82(12): p. 5887-911.
6. Asokan, A., et al., Reengineering a receptor footprint of adeno-associated virus enables selective and systemic gene transfer to muscle. Nat Biotechnol, 2010. 28(1): p. 79-82.
7. Pulicherla, N., et al., Engineering liver-detargeted AAV9 vectors for cardiac and musculoskeletal gene transfer. Mol Ther, 2011. 19(6): p. 1070-8.
8. DiMattia, M. A., et al., Structural insight into the unique properties of adeno-associated virus serotype 9. J Virol, 2012. 86(12): p. 6947-58.
9. Li, C., et al., Single amino acid modification of adeno-associated virus capsid changes transduction and humoral immune profiles. J Virol, 2012. 86(15): p. 7752-9.
10. Raupp, C., et al., The threefold protrusions of adeno-associated virus type 8 are involved in cell surface targeting as well as postattachment processing. J Virol, 2012. 86(17): p. 9396-408.
11. Govindasamy, L., et al., Structural insights into adeno-associated virus serotype 5. J Virol, 2013. 87(20): p. 11187-99.
12. Bentzinger, C. F., et al., Cellular dynamics in the muscle satellite cell niche. EMBO Rep, 2013. 14(12): p. 1062-72.
13. Wang, Y. X., N. A. Dumont, and M. A. Rudnicki, Muscle stem cells at a glance. J Cell Sci, 2014. 127(21): p. 4543-8.
14. Loiler, S. A., et al., Targeting recombinant adeno-associated virus vectors to enhance gene transfer to pancreatic islets and liver. Gene Ther, 2003. 10(18): p. 1551-8.

| SEQ ID NO 1 | | |
|---|---|---|
| Codon-optimized GALT sequence | | |
| 1 | ATGAGCAGAA GCGGCACCGA CCCTCAGCAG AGACAGCAGG CCTCTGAAGC CGATGCCGCC | |
| 61 | GCTGCCACCT TCAGAGCCAA TGACCACCAG CACATCCGGT ACAACCCCCT GCAGGACGAG | |
| 121 | TGGGTGCTGG TGTCCGCCCA CAGAATGAAG AGGCCTTGGC AGGGCCAGGT GGAACCCCAG | |
| 181 | CTGCTGAAAA CCGTGCCCAG ACACGACCCC CTGAACCCTC TGTGTCCTGG CGCCATTAGA | |
| 241 | GCCAACGGCG AAGTGAACCC CCAGTACGAC AGCACCTTCC TGTTCGACAA CGACTTCCCC | |
| 301 | GCCCTGCAGC CTGATGCCCC ATCTCCTGGA CCTAGCGACC ACCCTCTGTT CCAGGCCAAG | |
| 361 | TCTGCCAGAG GCGTGTGCAA AGTGATGTGC TTCCACCCTT GGAGCGACGT GACCCTGCCC | |
| 421 | CTGATGAGCG TGCCAGAGAT CAGAGCCGTG GTGGATGCCT GGGCCAGCGT GACAGAAGAA | |
| 481 | CTGGGAGCCC AGTACCCCTG GGTGCAGATC TTCGAGAACA AGGGCGCCAT GATGGGCTGC | |
| 541 | AGCAACCCCC ACCCTCACTG TCAAGTGTGG GCCAGCAGCT TCCTGCCCGA TATCGCCCAG | |
| 601 | CGGGAAGAGA GAAGCCAGCA GGCTTACAAG AGCCAGCACG GCGAGCCCCT GCTGATGGAA | |
| 661 | TACTCCAGAC AGGAACTGCT GCGGAAAGAA CGGCTGGTGC TGACCAGCGA GCACTGGCTG | |
| 721 | GTGCTGGTGC CTTTTTGGGC CACATGGCCC TACCAGACCC TGCTGCTGCC TAGAAGGCAC | |
| 781 | GTGCGGAGAC TGCCTGAGCT GACACCCGCC GAGAGAGATG ACCTGGCCAG CATCATGAAG | |
| 841 | AAACTGCTGA CCAAATACGA CAACCTGTTC GAGACCAGCT TCCCCTACAG CATGGGCTGG | |
| 901 | CACGGCGCTC CTACAGGATC TGAGGCTGGC GCCAACTGGA ACCACTGGCA GCTGCACGCC | |
| 961 | CACTACTACC CCCCACTGCT GAGATCTGCC ACCGTGCGGA AGTTCATGGT GGGATACGAG | |
| 1021 | ATGCTGGCTC AGGCCCAGAG AGATCTGACC CCTGAACAGG CCGCCGAACG GCTGAGAGCA | |
| 1081 | CTGCCCGAAG TGCACTACCA CCTGGGACAG AAGGACAGAG AGACAGCCAC AATCGCCTGA | |
| SEQ ID NO 2 - pAAV-CB-hGALT | | |
| Mutated ITR: Nucleotide (NT): 1-106 | | |
| CMV Enhancer: NT: 153-432 | | |
| Chicken Beta-Actin Promoter: NT 439-708 | | |
| Modified SV40 Intron: 774-833 | | |
| Human GALT: 1004-2143 | | |
| Bovine Growth Hormone Polyadenylation Signal: NT 2186-2332 | | |
| ITR: NT: 2412-2552 | | |
| Ampicillin Resistance Gene: NT 3455-4315 | | |
| Plasmid Origin of Replication (ori): NT: 4470-5089 | | |
| 1 | CTGCGCGCTC GCTCGCTCAC TGAGGCCGCC CGGGCAAAGC CCGGGCGTCG GGCGACCTTT | |
| 61 | GGTCGCCCGG CCTCAGTGAG CGAGCGAGCG CGCAGAGAGG GAGTGGAATT CACGCGTGGA | |
| 121 | TCTGAATTCA ATTCACGCGT GGTACCTCTG GTCGTTACAT AACTTACGGT AAATGGCCCG | |
| 181 | CCTGGCTGAC CGCCCAACGA CCCCCGCCCA TTGACGTCAA TAATGACGTA TGTTCCCATA | |
| 241 | GTAACGCCAA TAGGGACTTT CCATTGACGT CAATGGGTGG AGTATTTACG GTAAACTGCC | |
| 301 | CACTTGGCAG TACATCAAGT GTATCATATG CCAAGTACGC CCCCTATTGA CGTCAATGAC | |
| 361 | GGTAAATGGC CCGCCTGGCA TTATGCCCAG TACATGACCT TATGGGACTT TCCTACTTGG | |
| 421 | CAGTACATCT ACTCGAGGCC ACGTTCTGCT TCACTCTCCC CATCTCCCCC CCCTCCCCAC | |
| 481 | CCCCAATTTT GTATTTATTT ATTTTTTAAT TATTTTGTGC AGCGATGGGG GCGGGGGGGG | |
| 541 | GGGGGGGGCG CGCGCCAGGC GGGGCGGGGC GGGGCGAGGG GCGGGGCGGG GCGAGGCGGA | |
| 601 | GAGGTGCGGC GGCAGCCAAT CAGAGCGGCG CGCTCCGAAA GTTTCCTTTT ATGGCGAGGC | |
| 661 | GGCGGCGGCG GCGGCCCTAT AAAAAGCGAA GCGCGCGGCG GGCGGGAGCG GGATCAGCCA | |
| 721 | CCGCGGTGGC GGCCTAGAGT CGACGAGGAA CTGAAAAACC AGAAAGTTAA CTGGTAAGTT | |
| 781 | TAGTCTTTTT GTCTTTTATT TCAGGTCCCG GATCCGGTGG TGGTGCAAAT CAAAGAACTG | |
| 841 | CTCCTCAGTG GATGTTGCCT TTACTTCTAG GCCTGTACGG AAGTGTTACT TCTGCTCTAA | |
| 901 | AAGCTGCGGA ATTGTACCCG CGGCCGATCC ACCGGTCTTA AGGGCCGAGG CGGCCAGATC | |
| 961 | TTTCGAAGAT ATCGGCGCCG CTAGCGCGGC CGCAGCTGCC ACCATGAGCA GAAGCGGCAC | |
| 1021 | CGACCCTCAG CAGAGACAGC AGGCCTCTGA AGCCGATGCC GCCGCTGCCA CCTTCAGAGC | |
| 1081 | CAATGACCAC CAGCACATCC GGTACAACCC CCTGCAGGAC GAGTGGGTGC TGGTGTCCGC | |
| 1141 | CCACAGAATG AAGAGGCCTT GGCAGGGCCA GGTGGAACCC CAGCTGCTGA AAACCGTGCC | |
| 1201 | CAGACACGAC CCCCTGAACC CTCTGTGTCC TGGCGCCATT AGAGCCAACG GCGAAGTGAA | |
| 1261 | CCCCCAGTAC GACAGCACCT TCCTGTTCGA CAACGACTTC CCCGCCCTGC AGCCTGATGC | |
| 1321 | CCCATCTCCT GGACCTAGCG ACCACCCTCT GTTCCAGGCC AAGTCTGCCA GAGGCGTGTG | |
| 1381 | CAAAGTGATG TGCTTCCACC CTTGGAGCGA CGTGACCCTG CCCCTGATGA GCGTGCCAGA | |
| 1441 | GATCAGAGCC GTGGTGGATG CCTGGGCCAG CGTGACAGAA GAACTGGGAG CCCAGTACCC | |
| 1501 | CTGGGTGCAG ATCTTCGAGA ACAAGGGCGC CATGATGGGC TGCAGCAACC CCCACCCTCA | |
| 1561 | CTGTCAAGTG TGGGCCAGCA GCTTCCTGCC CGATATCGCC CAGCGGGAAG AGAGAAGCCA | |
| 1621 | GCAGGCTTAC AAGAGCCAGC ACGGCGAGCC CCTGCTGATG GAATACTCCA GACAGGAACT | |
| 1681 | GCTGCGGAAA GAACGGCTGG TGCTGACCAG CGAGCACTGG CTGGTGCTGG TGCCTTTTTG | |
| 1741 | GGCCACATGG CCCTACCAGA CCCTGCTGCT GCCTAGAAGG CACGTGCGGA GACTGCCTGA | |
| 1801 | GCTGACACCC GCCGAGAGAG ATGACCTGGC CAGCATCATG AAGAAACTGC TGACCAAATA | |
| 1861 | CGACAACCTG TTCGAGACCA GCTTCCCCTA CAGCATGGGC TGGCACGGCG CTCCTACAGG | |
| 1921 | ATCTGAGGCT GGCGCCAACT GGAACCACTG GCAGCTGCAC GCCCACTACT ACCCCCCACT | |
| 1981 | GCTGAGATCT GCCACCGTGC GGAAGTTCAT GGTGGGATAC GAGATGCTGG CTCAGGCCCA | |
| 2041 | GAGAGATCTG ACCCCTGAAC AGGCCGCCGA ACGGCTGAGA GCACTGCCCG AAGTGCACTA | |
| 2101 | CCACCTGGGA CAGAAGGACA GAGAGACAGC CACAATCGCC TGAAGTCAAG CTTATCGATA | |
| 2161 | CCGTCGACTA GAGCTCGCTG ATCAGCCTCG ACTGTGCCTT CTAGTTGCCA GCCATCTGTT | |
| 2221 | GTTTGCCCCT CCCCCGTGCC TTCCTTGACC CTGGAAGGTG CCACTCCCAC TGTCCTTTCC | |
| 2281 | TAATAAAATG AGGAAATTGC ATCGCATTGT CTGAGTAGGT GTCATTCTAT TCTGGGGGGT | |
| 2341 | GGGGTGGGGC AGGACAGCAA GGGGGAGGAT TGGGAAGACA ATAGCAGGCA TGCTGGGGAG | |
| 2401 | AGATCGATCT GAGGAACCCC TAGTGATGGA GTTGGCCACT CCCTCTCTGC GCGCTCGCTC | |
| 2461 | GCTCACTGAG GCCGGGCGAC CAAAGGTCGC CCGACGCCCG GGCTTTGCCC GGGCGGCCTC | |
| 2521 | AGTGAGCGAG CGAGCGCGCA GAGAGGGAGT GGCCCCCCCC CCCCCCCCCC CGGCGATTCT | |
| 2581 | CTTGTTTGCT CCAGACTCTC AGGCAATGAC CTGATAGCCT TTGTAGAGAC CTCTCAAAAA | |
| 2641 | TAGCTACCCT CTCCGGCATG AATTTATCAG CTAGAACGGT TGAATATCAT ATTGATGGTG | |
| 2701 | ATTTGACTGT CTCCGGCCTT TCTCACCCGT TTGAATCTTT ACCTACACAT TACTCAGGCA | |
| 2761 | TTGCATTTAA AATATATGAG GGTTCTAAAA ATTTTTATCC TTGCGTTGAA ATAAAGGCTT | |
| 2821 | CTCCCGCAAA AGTATTACAG GGTCATAATG TTTTTGGTAC AACCGATTTA GCTTTATGCT | |
| 2881 | CTGAGGCTTT ATTGCTTAAT TTTGCTAATT CTTTGCCTTG CCTGTATGAT TTATTGGATG | |
| 2941 | TTGGAATCGC CTGATGCGGT ATTTTCTCCT TACGCATCTG TGCGGTATTT CACACCGCAT | |
| 3001 | ATGGTGCACT CTCAGTACAA TCTGCTCTGA TGCCGCATAG TTAAGCCAGC CCCGACACCC | |
| 3061 | GCCAACACTA TGGTGCACTC TCAGTACAAT CTGCTCTGAT GCCGCATAGT TAAGCCAGCC | |
| 3121 | CCGACACCCG CCAACACCCG CTGACGCGCC CTGACGGGCT TGTCTGCTCC CGGCATCCGC | |
| 3181 | TTACAGACAA GCTGTGACCG TCTCCGGGAG CTGCATGTGT CAGAGGTTTT CACCGTCATC | |
| 3241 | ACCGAAACGC GCGAGACGAA AGGGCCTCGT GATACGCCTA TTTTTATAGG TTAATGTCAT | |
| 3301 | GATAATAATG GTTTCTTAGA CGTCAGGTGG CACTTTTCGG GGAAATGTGC GCGGAACCCC | |
| 3361 | TATTTGTTTA TTTTTCTAAA TACATTCAAA TATGTATCCG CTCATGAGAC AATAACCCTG | |
| 3421 | ATAAATGCTT CAATAATATT GAAAAAGGAA GAGTATGAGT ATTCAACATT TCCGTGTCGC | |
| 3481 | CCTTATTCCC TTTTTTGCGG CATTTTGCCT TCCTGTTTTT GCTCACCCAG AAACGCTGGT | |
| 3541 | GAAAGTAAAA GATGCTGAAG ATCAGTTGGG TGCACGAGTG GGTTACATCG AACTGGATCT | |
| 3601 | CAACAGCGGT AAGATCCTTG AGAGTTTTCG CCCCGAAGAA CGTTTTCCAA TGATGAGCAC | |
| 3661 | TTTTAAAGTT CTGCTATGTG GCGCGGTATT ATCCCGTATT GACGCCGGGC AAGAGCAACT | |
| 3721 | CGGTCGCCGC ATACACTATT CTCAGAATGA CTTGGTTGAG TACTCACCAG TCACAGAAAA | |
| 3781 | GCATCTTACG GATGGCATGA CAGTAAGAGA ATTATGCAGT GCTGCCATAA CCATGAGTGA | |
| 3841 | TAACACTGCG GCCAACTTAC TTCTGACAAC GATCGGAGGA CCGAAGGAGC TAACCGCTTT | |
| 3901 | TTTGCACAAC ATGGGGGATC ATGTAACTCG CCTTGATCGT TGGGAACCGG AGCTGAATGA | |
| 3961 | AGCCATACCA AACGACGAGC GTGACACCAC GATGCCTGTA GCAATGGCAA CAACGTTGCG | |
| 4021 | CAAACTATTA ACTGGCGAAC TACTTACTCT AGCTTCCCGG CAACAATTAA TAGACTGGAT | |
| 4081 | GGAGGCGGAT AAAGTTGCAG GACCACTTCT GCGCTCGGCC CTTCCGGCTG GCTGGTTTAT | |
| 4141 | TGCTGATAAA TCTGGAGCCG GTGAGCGTGG GTCTCGCGGT ATCATTGCAG CACTGGGGCC | |
| 4201 | AGATGGTAAG CCCTCCCGTA TCGTAGTTAT CTACACGACG GGGAGTCAGG CAACTATGGA | |
| 4261 | TGAACGAAAT AGACAGATCG CTGAGATAGG TGCCTCACTG ATTAAGCATT GGTAACTGTC | |
| 4321 | AGACCAAGTT TACTCATATA TACTTTAGAT TGATTTAAAA CTTCATTTTT AATTTAAAAG | |
| 4381 | GATCTAGGTG AAGATCCTTT TTGATAATCT CATGACCAAA ATCCCTTAAC GTGAGTTTTC | |
| 4441 | GTTCCACTGA GCGTCAGACC CCGTAGAAAA GATCAAAGGA TCTTCTTGAG ATCCTTTTTT | |
| 4501 | TCTGCGCGTA ATCTGCTGCT TGCAAACAAA AAAACCACCG CTACCAGCGG TGGTTTGTTT | |
| 4561 | GCCGGATCAA GAGCTACCAA CTCTTTTTCC GAAGGTAACT GGCTTCAGCA GAGCGCAGAT | |
| 4621 | ACCAAATACT GTTCTTCTAG TGTAGCCGTA GTTAGGCCAC CACTTCAAGA ACTCTGTAGC | |
| 4681 | ACCGCCTACA TACCTCGCTC TGCTAATCCT GTTACCAGTG GCTGCTGCCA GTGGCGATAA | |
| 4741 | GTCGTGTCTT ACCGGGTTGG ACTCAAGACG ATAGTTACCG GATAAGGCGC AGCGGTCGGG | |
| 4801 | CTGAACGGGG GGTTCGTGCA CACAGCCCAG CTTGGAGCGA ACGACCTACA CCGAACTGAG | |
| 4861 | ATACCTACAG CGTGAGCTAT GAGAAAGCGC CACGCTTCCC GAAGGGAGAA AGGCGGACAG | |
| 4921 | GTATCCGGTA AGCGGCAGGG TCGGAACAGG AGAGCGCACG AGGGAGCTTC CAGGGGGAAA | |
| 4981 | CGCCTGGTAT CTTTATAGTC CTGTCGGGTT TCGCCACCTC TGACTTGAGC GTCGATTTTT | |
| 5041 | GTGATGCTCG TCAGGGGGGC GGAGCCTATG GAAAAACGCC AGCAACGCGG CCTTTTTACG | |
| 5101 | GTTCCTGGCC TTTTGCTGGC CTTTTGCTCA CATGTTCTTT CCTGCGTTAT CCCCTGATTC | |
| 5161 | TGTGGATAAC CGTATTACCG CCTTTGAGTG AGCTGATACC GCTCGCCGCA GCCGAACGAC | |
| 5221 | CGAGCGCAGC GAGTCAGTGA GCGAGGAAGC GGAAGAGCGC CCAATACGCA AACCGCCTCT | |
| 5281 | CCCCGCGCGT TGGCCGATTC ATTAATGCAG CTGGCGTAAT AGCGAAGAGG CCCGCACCGA | |
| 5341 | TCGCCCTTCC CAACAGTTGC GCAGCCTGAA TGGCGAATGG CGATTCCGTT GCAATGGCTG | |
| 5401 | GCGGTAATAT TGTTCTGGAT ATTACCAGCA AGGCCGATAG TTTGAGTTCT TCTACTCAGG | |
| 5461 | CAAGTGATGT TATTACTAAT CAAAGAAGTA TTGCGACAAC GGTTAATTTG CGTGATGGAC | |
| 5521 | AGACTCTTTT ACTCGGTGGC CTCACTGATT ATAAAAACAC TTCTCAGGAT TCTGGCGTAC | |
| 5581 | CGTTCCTGTC TAAAATCCCT TTAATCGGCC TCCTGTTTAG CTCCCGCTCT GATTCTAACG | |
| 5641 | AGGAAAGCAC GTTATACGTG CTCGTCAAAG CAACCATAGT ACGCGCCCTG TAGCGGCGCA | |
| 5701 | TTAAGCGCGG CGGGTGTGGT GGTTACGCGC AGCGTGACCG CTACACTTGC CAGCGCCCTA | |
| 5761 | GCGCCCGCTC CTTTCGCTTT CTTCCCTTCC TTTCTCGCCA CGTTCGCCGG CTTTCCCCGT | |
| 5821 | CAAGCTCTAA ATCGGGGGCT CCCTTTAGGG TTCCGATTTA GTGCTTTACG GCACCTCGAC | |
| 5881 | CCCAAAAAAC TTGATTAGGG TGATGGTTCA CGTAGTGGGC CATCGCCCTG ATAGACGGTT | |
| 5941 | TTTCGCCCTT TGACGTTGGA GTCCACGTTC TTTAATAGTG GACTCTTGTT CCAAACTGGA | |
| 6001 | ACAACACTCA ACCCTATCTC GGTCTATTCT TTTGATTTAT AAGGGATTTT GCCGATTTCG | |
| 6061 | GCCTATTGGT TAAAAAATGA GCTGATTTAA CAAAAATTTA ACGCGAATTT TAACAAAATA | |
| 6121 | TTAACGCTTA CAATTTAAAT ATTTGCTTAT ACAATCTTCC TGTTTTTGGG GCTTTTCTGA | |
| 6181 | TTATCAACCG GGGTACATAT GATTGACATG CTAGTTTTAC GATTACCGTT CATCGCC | |
| SEQ ID NO 3: pAAV-CB-hGALT-WPREv2-KAN | | |
| Mutated ITR: Nucleotide (NT): 1-145 | | |
| CMV Enhancer: NT: 190-551 | | |
| Chicken Beta-Actin Promoter: NT 552-834 | | |
| Chimeric Intron: 929-1103 | | |
| Human GALT: 1160-2299 | | |
| Woodchuck Hepatits Virus Pos Response Element (WPRE): NT 2334-2927 | | |
| Poly A Signal (BGHpA): NT 2942-3176 | | |
| ITR: NT: 3449-3592 | | |
| KAN Gene: NT 5145-5960 | | |
| 1 | GCCCAATACG CAAACCGCCT CTCCCCGCGC GTTGGCCGAT TCATTAATGC AGCTGGCGCG | |
| 61 | CTCGCTCGCT CACTGAGGCC GCCCGGGCAA AGCCCGGGCG TCGGGCGACC TTTGGTCGCC | |
| 121 | CGGCCTCAGT GAGCGAGCGA GCGCGCAGAG AGGGAGTGGC CAACTCCATC ACTAGGGGTT | |
| 181 | CCTTGTAGTT AATGATTAAC CCGCCATGCT AATTATCTAC GTAGCCATGT CTAGACAGCC | |
| 241 | ACTATGGGTC TAGGCTGCCC ATGTAAGGAG GCAAGGCCTA GTTATTAATA GTAATCAATT | |
| 301 | ACGGGGTCAT TAGTTCATAG CCCATATATG GAGTTCCGCG TTACATAACT TACGGTAAAT | |
| 361 | GGCCCGCCTG GCTGACCGCC CAACGACCCC CGCCCATTGA CGTCAATAAT GACGTATGTT | |
| 421 | CCCATAGTAA CGCCAATAGG GACTTTCCAT TGACGTCAAT GGGTGGACTA TTTACGGTAA | |
| 481 | ACTGCCCACT TGGCAGTACA TCAAGTGTAT CATATGCCAA GTACGCCCCC TATTGACGTC | |
| 541 | AATGACGGTA AATGGCCCGC CTGGCATTAT GCCCAGTACA TGACCTTATG GGACTTTCCT | |
| 601 | ACTTGGCAGT ACATCTACGT ATTAGTCATC GCTATTACCA TGGTCGAGGT GAGCCCCACG | |
| 661 | TTCTGCTTCA CTCTCCCCAT CTCCCCCCCC TCCCCACCCC CAATTTTGTA TTTATTTATT | |
| 721 | TTTTAATTAT TTTGTGCAGC GATGGGGGCG GGGGGGGGGG GGGGGCGCGC GCCAGGCGGG | |
| 781 | GCGGGGCGGG GCGAGGGGCG GGGCGGGGCG AGGCGGAGAG GTGCGGCGGC AGCCAATCAG | |
| 841 | AGCGGCGCGC TCCGAAAGTT TCCTTTTATG GCGAGGCGGC GGCGGCGGCG GCCCTATAAA | |
| 901 | AAGCGAAGCG CGCGGCGGGC GGGAGTCGCT GCGACGCTGC CTTCGCCCCG TGCCCCGCTC | |
| 961 | CGCCGCCGCC TCGCGCCGCC CGCCCCGGCT CTGACTGACC GCGTTACTCC CACAGGTGAG | |
| 1021 | CGGGCGGGAC GGCCCTTCTC CTCCGGGCTG TAATTAGCGC TTGGTTTAAT GACGGCTTGT | |
| 1081 | TTCTTTTCTG TGGCTGCGTG AAAGCCTTGA GGGGCTCCGG GAGCTAGAGC CTCTGCTAAC | |
| 1141 | CATGTTCATG CCTTCTTCTT TTTCCTACAG CTCCTGGGCA ACGTGCTGGT TATTGTGCTG | |
| 1201 | TCTCATCATT TTGGCAAAGA ATTCTAGCGC GGCCGCAGCT GCCACCATGA GCAGAAGCGG | |
| 1261 | CACCGACCCT CAGCAGAGAC AGCAGGCCTC TGAAGCCGAT GCCGCCGCTG CCACCTTCAG | |
| 1321 | AGCCAATGAC CACCAGCACA TCCGGTACAA CCCCCTGCAG GACGAGTGGG TGCTGGTGTC | |
| 1381 | CGCCCACAGA ATGAAGAGGC CTTGGCAGGG CCAGGTGGAA CCCCAGCTGC TGAAAACCGT | |
| 1441 | GCCCAGACAC GACCCCCTGA ACCCTCTGTG TCCTGGCGCC ATTAGAGCCA ACGGCGAAGT | |
| 1501 | GAACCCCCAG TACGACAGCA CCTTCCTGTT CGACAACGAC TTCCCCGCCC TGCAGCCTGA | |
| 1561 | TGCCCCATCT CCTGGACCTA GCGACCACCC TCTGTTCCAG GCCAAGTCTG CCAGAGGCGT | |
| 1621 | GTGCAAAGTG ATGTGCTTCC ACCCTTGGAG CGACGTGACC CTGCCCCTGA TGAGCGTGCC | |
| 1681 | AGAGATCAGA GCCGTGGTGG ATGCCTGGGC CAGCGTGACA GAAGAACTGG GAGCCCAGTA | |
| 1741 | CCCCTGGGTG CAGATCTTCG AGAACAAGGG CGCCATGATG GGCTGCAGCA ACCCCCACCC | |
| 1801 | TCACTGTCAA GTGTGGGCCA GCAGCTTCCT GCCCGATATC GCCCAGCGGG AAGAGAGAAG | |
| 1861 | CCAGCAGGCT TACAAGAGCC AGCACGGCGA GCCCCTGCTG ATGGAATACT CCAGACAGGA | |
| 1921 | ACTGCTGCGG AAAGAACGGC TGGTGCTGAC CAGCGAGCAC TGGCTGGTGC TGGTGCCTTT | |
| 1981 | TTGGGCCACA TGGCCCTACC AGACCCTGCT GCTGCCTAGA AGGCACGTGC GGAGACTGCC | |
| 2041 | TGAGCTGACA CCCGCCGAGA GAGATGACCT GGCCAGCATC ATGAAGAAAC TGCTGACCAA | |
| 2101 | ATACGACAAC CTGTTCGAGA CCAGCTTCCC CTACAGCATG GGCTGGCACG GCGCTCCTAC | |
| 2161 | AGGATCTGAG GCTGGCGCCA ACTGGAACCA CTGGCAGCTG CACGCCCACT ACTACCCCCC | |
| 2221 | ACTGCTGAGA TCTGCCACCG TGCGGAAGTT CATGGTGGGA TACGAGATGC TGGCTCAGGC | |
| 2281 | CCAGAGAGAT CTGACCCCTG AACAGGCCGC CGAACGGCTG AGAGCACTGC CCGAAGTGCA | |
| 2341 | CTACCACCTG GGACAGAAGG ACAGAGAGAC AGCCACAATC GCCTGAAGTC AAGCTTATCG | |
| 2401 | ATAATCAACC TCTGGATTAC AAAATTTGTG AAAGATTGAC TGGTATTCTT AACTATGTTG | |
| 2461 | CTCCTTTTAC GCTATGTGGA TACGCTGCTT TAATGCCTTT GTATCATGCT ATTGCTTCCC | |
| 2521 | GTATGGCTTT CATTTTCTCC TCCTTGTATA AATCCTGGTT GCTGTCTCTT TATGAGGAGT | |
| 2581 | TGTGGCCCGT TGTCAGGCAA CGTGGCGTGG TGTGCACTGT GTTTGCTGAC GCAACCCCCA | |
| 2641 | CTGGTTGGGG CATTGCCACC ACCTGTCAGC TCCTTTCCGG GACTTTCGCT TTCCCCCTCC | |
| 2701 | CTATTGCCAC GGCGGAACTC ATCGCCGCCT GCCTTGCCCG CTGCTGGACA GGGGCTCGGC | |
| 2761 | TGTTGGGCAC TGACAATTCC GTGGTGTTGT CGGGGAAATC ATCGTCCTTT CCTTGGCTGC | |
| 2821 | TCGCCTGTGT TGCCACCTGG ATTCTGCGCG GGACGTCCTT CTGCTACGTC CCTTCGGCCC | |
| 2881 | TCAATCCAGC GGACCTTCCT TCCCGCGGCC TGCTGCCGGC TCTGCGGCCT CTTCCGCGTC | |
| 2941 | TTCGCCTTCG CCCTCAGACG AGTCGGATCT CCCTTTGGGC CGCCTCCCCG CATCGATACC | |
| 3001 | GTCGAGGCCG CAATAAAAGA TCTTTATTTT CATTAGATCT GTGTGTTGGT TTTTTGTGTG | |
| 3061 | TCTAGACATG GCTACGTAGA TAATTAGCAT GGCGGGTTAA TCATTAACTA CAAGGAACCC | |
| 3121 | CTAGTGATGG AGTTGGCCAC TCCCTCTCTG CGCGCTCGCT CGCTCACTGA GGCCGGGCGA | |
| 3181 | CCAAAGGTCG CCCGACGCCC GGGCTTTGCC CGGGCGGCCT CAGTGAGCGA GCGAGCGCGC | |
| 3241 | CAGCTGGCGT AATAGCGAAG AGGCCCGCAC CGATCGCCCT TCCCAACAGT TGCGCAGCCT | |
| 3301 | GAATGGCGAA TGGAAGTTCC GTTGCAATGG CTGGCGGTAA TATTGTTCTG GATATTACCA | |
| 3361 | GCAAGGCCGA TAGTTTGAGT TCTTCTACTC AGGCAAGTGA TGTTATTACT AATCAAAGAA | |
| 3421 | GTATTGCGAC AACGGTTAAT TTGCGTGATG GACAGACTCT TTTACTCGGT GGCCTCACTG | |
| 3481 | ATTATAAAAA CACTTCTCAG GATTCTGGCG TACCGTTCCT GTCTAAAATC CCTTTAATCG | |
| 3541 | GCCTCCTGTT TAGCTCCCGC TCTGATTCTA ACGAGGAAAG CACGTTATAC GTGCTCGTCA | |
| 3601 | AAGCAACCAT AGTACGCGCC CTGTAGCGGC GCATTAAGCG CGGCGGGTGT GGTGGTTACG | |
| 3661 | CGCAGCGTGA CCGCTACACT TGCCAGCGCC CTAGCGCCCG CTCCTTTCGC TTTCTTCCCT | |
| 3721 | TCCTTTCTCG CCACGTTCGC CGGCTTTCCC CGTCAAGCTC TAAATCGGGG GCTCCCTTTA | |
| 3781 | GGGTTCCGAT TTAGTGATTT ACGGCACCTC GACCCCAAAA AACTTGATTA GGGTGATGGT | |
| 3841 | TCACGTAGTG GGCCATCGCC CTGATAGACG GTTTTTCGCC CTTTGACGTT GGAGTCCACG | |
| 3901 | TTCTTTAATA GTGGACTCTT GTTCCAAACT GGAACAACAC TCAACCCTAT CTCGGTCTAT | |
| 3961 | TCTTTTGATT TATAAGGGAT TTTGCCGATT TCGGCCTATT GGTTAAAAAA TGAGCTGATT | |
| 4021 | TAACAAAAAT TTAACGCGAA TTTTAACAAA ATATTAACGT TTACAATTTA AATATTTGCT | |
| 4081 | TATACAATCT TCCTGTTTTT GGGGCTTTTC TGATTATCAA CCGGGGTACA TATGATTGAC | |
| 4141 | ATGCTAGTTT TACGATTACC GTTCATCGAT TCTCTTGTTT GCTCCAGACT CTCAGGCAAT | |
| 4201 | GACCTGATAG CCTTTGTAGA GACCTCTCAA AAATAGCTAC CCTCTCCGGC ATGAATTTAT | |
| 4261 | CAGCTAGAAC GGTTGAATAT CATATTGATG GTGATTTGAC TGTCTCCGGC CTTTCTCACC | |
| 4321 | CGTTTGAATC TTTACCTACA CATTACTCAG GCATTGCATT TAAAATATAT GAGGGTTCTA | |
| 4381 | AAAATTTTTA TCCTTGCGTT GAAATAAAGG CTTCTCCCGC AAAAGTATTA CAGGGTCATA | |
| 4441 | ATGTTTTTGG TACAACCGAT TTAGCTTTAT GCTCTGAGGC TTTATTGCTT AATTTTGCTA | |
| 4501 | ATTCTTTGCC TTGCCTGTAT GATTTATTGG ATGTTGGAAG TTCCTGATGC GGTATTTTCT | |
| 4561 | CCTTACGCAT CTGTGCGGTA TTTCACACCG CATATGGTGC ACTCTCAGTA CAATCTGCTC | |
| 4621 | TGATGCCGCA TAGTTAAGCC AGCCCCGACA CCCGCCAACA CCCGCTGACG CGCCCTGACG | |
| 4681 | GGCTTGTCTG CTCCCGGCAT CCGCTTACAG ACAAGCTGTG ACCGTCTCCG GGAGCTGCAT | |
| 4741 | GTGTCAGAGG TTTTCACCGT CATCACCGAA ACGCGCGAGA CGAAAGGGCC TCGTGATACG | |
| 4801 | CCTATTTTTA TAGGTTAATG TCATGATAAT AATGGTTTCT TAGACGTCAG GTGGCACTTT | |
| 4861 | TCGGGGAAAT GTGCGCGGAA CCCCTATTTG TTTATTTTTC TAAATACATT CAAATATGTA | |
| 4921 | TCCGCTCATG AGACAATAAC CCTGATAAAT GCTTCAATAA TATTGAAAAA GGAAGAGTAT | |
| 4981 | GAGTATTCAA CATTTCCGTG TCGCCCTTAT TCCCTTTTTT GCGGCATTTT GCCTTCCTGT | |
| 5041 | TTTTGCTCAC CCAGAAACGC TGGTGAAAGT AAAAGATGCT GAAGATCAGT TGGGTGCACG | |
| 5101 | AGTGGGTTAC ATCGAACTGG ATCTCAACAG CGGTAAGATC CTTGAGAGTT TTCGCCCCGA | |
| 5161 | AGAACGTTTT CCAATGATGA GCACTTTTAA AGTTCTGCTA TGTGGCGCGG TATTATCCCG | |
| 5221 | TATTGACGCC GGGCAAGAGC AACTCGGTCG CCGCATACAC TATTCTCAGA ATGACTTGGT | |
| 5281 | TGAGTACTCA CCAGTCACAG AAAAGCATCT TACGGATGGC ATGACAGTAA GAGAATTATG | |
| 5341 | CAGTGCTGCC ATAACCATGA GTGATAACAC TGCGGCCAAC TTACTTCTGA CAACGATCGG | |
| 5401 | AGGACCGAAG GAGCTAACCG CTTTTTTGCA CAACATGGGG GATCATGTAA CTCGCCTTGA | |
| 5461 | TCGTTGGGAA CCGGAGCTGA ATGAAGCCAT ACCAAACGAC GAGCGTGACA CCACGATGCC | |
| 5521 | TGTAGCAATG GCAACAACGT TGCGCAAACT ATTAACTGGC GAACTACTTA CTCTAGCTTC | |
| 5581 | CCGGCAACAA TTAATAGACT GGATGGAGGC GGATAAAGTT GCAGGACCAC TTCTGCGCTC | |
| 5641 | GGCCCTTCCG GCTGGCTGGT TTATTGCTGA TAAATCTGGA GCCGGTGAGC GTGGGTCTCG | |
| 5701 | CGGTATCATT GCAGCACTGG GGCCAGATGG TAAGCCCTCC CGTATCGTAG TTATCTACAC | |
| 5761 | GACGGGGAGT CAGGCAACTA TGGATGAACG AAATAGACAG ATCGCTGAGA TAGGTGCCTC | |
| 5821 | ACTGATTAAG CATTGGTAAC TGTCAGACCA AGTTTACTCA TATATACTTT AGATTGATTT | |
| 5881 | AAAACTTCAT TTTTAATTTA AAAGGATCTA GGTGAAGATC CTTTTTGATA ATCTCATGAC | |
| 5941 | CAAAATCCCT TAACGTGAGT TTTCGTTCCA CTGAGCGTCA GACCCCGTAG AAAAGATCAA | |
| 6001 | AGGATCTTCT TGAGATCCTT TTTTTCTGCG CGTAATCTGC TGCTTGCAAA CAAAAAAACC | |
| 6061 | ACCGCTACCA GCGGTGGTTT GTTTGCCGGA TCAAGAGCTA CCAACTCTTT TTCCGAAGGT | |
| 6121 | AACTGGCTTC AGCAGAGCGC AGATACCAAA TACTGTCCTT CTAGTGTAGC CGTAGTTAGG | |
| 6181 | CCACCACTTC AAGAACTCTG TAGCACCGCG TACATACCTC GCTCTGCTAA TCCTGTTACC | |
| 6241 | AGTGGCTGCT GCCAGTGGCG ATAAGTCGTG TCTTACCGGG TTGGACTCAA GACGATAGTT | |
| 6301 | ACCGGATAAG GCGCAGCGGT CGGGCTGAAC GGGGGGTTCG TGCACACAGC CCAGCTTGGA | |
| 6361 | GCGAACGACC TACACCGAAC TGAGATACCT ACAGCGTGAG CTATGAGAAA GCGCCACGCT | |
| 6421 | TCCCGAAGGG AGAAAGGCGG ACAGGTATCC GGTAAGCGGC AGGGTCGGAA CAGGAGAGCG | |
| 6481 | CACGAGGGAG CTTCCAGGGG GAAACGCCTG GTATCTTTAT AGTCCTGTCG GGTTTCGCCA | |
| 6541 | CCTCTGACTT GAGCGTCGAT TTTTGTGATG CTCGTCAGGG GGGCGGAGCC TATGGAAAAA | |
| 6601 | CGCCAGCAAC GCGGCCTTTT TACGGTTCCT GGCCTTTTGC TGGCCTTTTG CTCACATGTT | |
| 6661 | CTTTCCTGCG TTATCCCCTG ATTCTGTGGA TAACCGTATT ACCGGGTTTG AGTGAGCTGA | |
| 6721 | TACCGCTCGC CGCAGCCGAA CGACCGAGCG CAGCGAGTCA GTGAGCGACC AAGCGGAAGA | |
| 6781 | GC | |
| SEQ ID NO 4: | | |
| MSRSGTDPQQRQQASEADAAAATFRANDHQHIRYNPLQDEWVLVSAHRMKRPWQ | | |
| GQVEPQLLKTVPRHDPLNPLCPGAIRANGEVNPQYDSTFLFDNDFPALQPDAPSPGPS | | |
| DHPLFQAKSARGVCKVMCFHPWSDVTLPLMSVPEIRAVVDAWASVTEELGAQYPW | | |
| VQIFENKGAMMGCSNPHPHCQVWASSFLPDIAQREERSQQAYKSQHGEPLLMEYSR | | |
| QELLRKERLVLTSEHWLVLVPFWATWPYQTLLLPRRHVRRLPELTPAERDDLASIMK | | |
| KLLTKYDNLFETSFPYSMGWHGAPTGSEAGANWNHWQLHAHYYPPLLRSATVRKF | | |
| MVGYEMLAQAQRDLTPEQAAERLRALPEVHYHLGQKDRETATIA | | |
| SEQ ID NO 5: | | |
| Rh74 VP1 amino acid sequence | | |
| MAADGYLPDWLEDNLSEGIREWWDLKPGAPKPKANQQKQDNGRGLVLPGYKYLG | | |
| PFNGLDKGEPVNAADAAALEHDKAYDQQLQAGDNPYLRYNHADAEFQERLQEDTS | | |
| FGGNLGRAVFQAKKRVLEPLGLVESPVKTAPGKKRPVEPSPQRSPDSSTGIGKKGQQ | | |
| PAKKRLNFGQTGDSESVPDPQPIGEPPAGPSGLGSGTMAAGGGAPMADNNEGADGV | | |
| GSSSGNWHCDSTWLGDRVITTSTRTWALPTYNNHLYKQISNGTSGGSTNDNTYFGY | | |
| STPWGYFDFNRFHCHFSPRDWQRLINNNWGFRPKRLNFKLFNIQVKEVTQNEGTKTI | | |
| ANNLTSTIQVFTDSEYQLPYVLGSAHQGCLPPFPADVFMIPQYGYLTL | | |
| NNGSQAVGRSSFYCLEYFPSQMLRTGNNFEFSYNFEDVPFHSSYAHSQSLDRLMNPL | | |
| IDQYLYYLSRTQSTGGTAGTQQLLFSQAGPNNMSAQAKNWLPGPCYRQQRVSTTLS | | |
| QNNNSNFAWTGATKYHLNGRDSLVNPGVAMATHKDDEERFFPSSGVLMFGKQGA | | |
| GKDNVDYSSVMLTSEEEIKTTNPVATEQYGVVADNLQQQNAAP1VGAVNSQGALP | | |
| GMVWQNRDVYLQGPIWAK1PHTDGNFHPSPLMGGFGLKHPPPQILIKNTPVPADPPT | | |
| TFNQAKLASFITQYSTGQVSVEIEWELQKENSKRWNPEIQYTSNYYKSTNVDFAVNT | | |
| EGTYSEPRPIGTRYLTRNL | | |
| SEQ ID NO: 6 | | |
| Rh74 VP1 DNA | | |
| ATGGCTGCCGATGGTTATCTTCCAGATTGGCTCGAGGACAACCTCTCTGAGGGCA | | |
| TTCGCGAGTGGTGGGACCTGAAACCTGGAGCCCCGAAACCCAAAGCCAACCAGC | | |
| AAAAGCAGGACAACGGCCGGGGTCTGGTGCTTCCTGGCTACAAGTACCTCGGAC | | |
| CCTTCAACGGACTCGACAAGGGGGAGCCCGTCAACGCGGCGGACGCAGCGGCCC | | |
| TCGAGCACGACAAGGCCTACGACCAGCAGCTCCAAGCGGGTGACAATCCGTACC | | |
| TGCGGTATAATCACGCCGACGCCGAGTTTCAGGAGCGTCTGCAAGAAGATACGT | | |
| CTTTTGGGGGCAACCTCGGGCGCGCAGTCTTCCAGGCCAAAAAGCGGGTTCTCG | | |
| AACCTCTGGGCCTGGTTGAATCGCCGGTTAAGACGGCTCCTGGAAAGAAGAGAC | | |
| CGGTAGAGCCATCACCCCAGCGCTCTCCAGACTCCTCTACGGGCATCGGCAAGA | | |
| AAGGCCAGCAGCCCGCAAAAAAGAGACTCAATTTTGGGCAGACTGGCGACTCAG | | |
| AGTCAGTCCCCGACCCTCAACCAATCGGAGAACCACCAGCAGGCCCCTCTGGTCT | | |
| GGGATCTGGTACAATGGCTGCAGGCGGTGGCGCTCCAATGGCAGACAATAACGA | | |
| AGGCGCCGACGGAGTGGGTAGTTCCTCAGGAAATTGGCATTGCGATTCCACATG | | |
| GCTGGGCGACAGAGTCATCACCACCAGCACCCGCACCTGGGCCCTGCCCACCTA | | |
| CAACAACCACCTCTACAAGCAAATCTCCAACGGGACCTCGGGAGGAAGCACCAA | | |
| CGACAACACCTACTTCGGCTACAGCACCCCCTGGGGGTATTTTGACTTCAACAGA | | |
| TTCCACTGCCACTTTTCACCACGTGACTGGAGCGACTCATCAACAACAACTGGGG | | |
| ATTCCGGCCCAAGAGGCTCAACTTCAAGCTCTTCAACATCCAAGTCAAGGAGGTC | | |
| ACGCAGAATGAAGGCACCAAGAGCATCGCCAATAACCTTACCAGCAGGATTCAG | | |
| GTCTTTACGGACTCGGAATACCAGCTCCCGTACGTGCTCGGCTCGGCGCACCAGG | | |
| GCTGCCTGCCTCCGTTCCCGGCGGACGTCTTCATGATTCCTCAGTACGGGTACCT | | |
| GACTCTGAACAATGGCAGTCAGGCTGTGGGCCGGTCGTCCTTCTACTGCCTGGAG | | |
| TACTTTCCTTCTCAAATGCTGAGAACGGGCAACAACTTTGAATTCAGCTACAACT | | |
| TCGAGGACGTGCCCTTCCACAGCAGCTACGCGCACAGCCAGAGCCTGGACCGGC | | |
| TGATGAACCCTCTCATCGACCAGTACTTGTACTACCTGTCCCGGACTCAAAGCAC | | |
| GGGCGGTACTGCAGGAACTCAGCAGTTGCTATTTTCTCAGGCCGGGCCTAACAAC | | |
| ATGTCGGCTCAGGCCAAGAACTGGCTACCCGGTCCCTGCTACCGGCAGCACGCG | | |
| TCTCCACGACACTGTCGCAGAACAACAACAGCAACTTTGCCTGGACGGGTGCCA | | |
| CCAAGTATCATCTGAATGGCAGAGACTCTCTGGTGAATCCTGGCGTTGCCATGGC | | |
| TACCCACAAGGACGACGAAGAGCGATTTTTTCCATCCAGCGGAGTCTTAATGTTT | | |
| GGGAAACAGGGAGCTGGAAAAGACAACGTGGACTATAGCAGCGTGATGCTAAC | | |
| CAGCGAGGAAGAAATAAAGACCACCAACCCAGTGGCCACAGAACAGTACGGCG | | |
| TGGTGGCCGATAACCTGCAACAGCAAAACGCCGCTCCTATTGTAGGGGCCGTCA | | |
| ATAGTCAAGGAGCCTTACCTGGCATGGTGTGGCAGAACCGGGACGTGTACCTGC | | |
| AGGGTCCCATCTGGGCCAAGATTCCTCATACGGACGGCAACTTTCATCCCTCGCC | | |
| GCTGATGGGAGGCTTTGGACTGAAGCATCCGCCTCCTCAGATCCTGATTAAAAAC | | |
| ACACCTGTTCCCGCGGATCCTCCGACCACCTTCAATCAGGCCAAGCTGGCTTCTT | | |
| TCATCACGCAGTACAGTACCGGCCAGGTCAGCGTGGAGATCGAGTGGGAGCTGC | | |
| AGAAGGAGAACAGCAAACGCTGGAACCCAGAGATTCAGTACACTTCCAACTACT | | |
| ACAAATCTACAAATGTGGACTTTGCTGTCAATACTGAGGGTACTTATTCCGAGCC | | |
| TCGCCCCATTGGCACCCGTTACCTCACCCGTAATCTGTAA | | |
| SEQ ID NO: 7 - KAN Gene Translation Product | | |
| MSHIQRETSCSRPRLNSNMDADLYGYKWARDNVGQSGATIYRLYGKPDAPELFLKH | | |
| GKGSVANDVTDEMVRLNWLTEFMPLPTIKHFIRTPDDAWLLTTAIPGKTAFQVLEEY | | |
| PDSGENIVDALAVFLRRLHSIPVCNCPFNSDRVFRLAQAQSRMNNGLVDASDFDDER | | |
| NGWPVEQVWKEMHKLLPFSPDSVVTHGDFSLDNLIFDEGKLIGCIDVGRVGIADRYQ | | |
| DLAILWNCLGEFSPSLQKRLFQKYGIDNPDMNKLQFHLMLDEFF | | |
| AAV rh74 Consensus Sequence Alignment | | |
| AAVrh74 | | |
| AAVrh74-N5021-capsid | | |
| Rh74 YIG591 cap protein | | |
| Consensus | | |
| 1. AAVrh74 | | |
| 2. AAVrh-N5021-capsid | | |
| 3. rh74 YIG591 cap protein | | |
| | ********************************************************************** | |
| | MAADGYLPDWLEDNLSEGIREWWDLKPGAPKPKANQQKQDNGRGLVLPGYKYLGPFNGLDKGEPVNAADA | |
| | 10 20 30 40 50 60 70 | |
| 1 | MAADGYLPDWLEDNLSEGIREWWDLKPGAPKPKANQQKQDNGRGLVLPGYKYLGPFNGLDKGEPVNAADA | 70 |
| 2 | MAADGYLPDWLEDNLSEGIREWWDLKPGAPKPKANQQKQDNGRGLVLPGYKYLGPFNGLDKGEPVNAADA | 70 |
| 3 | MAADGYLPDWLEDNLSEGIREWWDLKPGAPKPKANQQKQDNGRGLVLPGYKYLGPFNGLDKGEPVNAADA | 70 |
| | ********************************************************************** | |
| | AALEHDKAYDQQLQAGDNPYLRYNHADAEFQERLQEDTSFGGNLGRAVFQAKKRVLEPLGLVESPVKTAP | |
| | 80 90 100 110 120 130 140 | |
| 1 | AALEHDKAYDQQLQAGDNPYLRYNHADAEFQERLQEDTSFGGNLGRAVFQAKKRVLEPLGLVESPVKTAP | 140 |
| 2 | AALEHDKAYDQQLQAGDNPYLRYNHADAEFQERLQEDTSFGGNLGRAVFQAKKRVLEPLGLVESPVKTAP | 140 |
| 3 | AALEHDKAYDQQLQAGDNPYLRYNHADAEFQERLQEDTSFGGNLGRAVFQAKKRVLEPLGLVESPVKTAP | 140 |
| | ********************************************************************** | |
| | GKKRPVEPSPQRSPDSSTGIGKKGQQPAKKRLNF [symbol] QTGDSESVPDPQPIGEPPAGPSGL [symbol] SGT [symbol] AAGGGA | |
| | 150 160 170 180 190 200 210 | |
| 1 | GKKRPVEPSPQRSPDSSTGIGKKGQQPAKKRLNF [symbol] QTGDSESVPDPQPIGEPPAGPSGL [symbol] SGT [symbol] AAGGGA | 210 |
| 2 | GKKRPVEPSPQRSPDSSTGIGKKGQQPAKKRLNF [symbol] QTGDSESVPDPQPIGEPPAGPSGL [symbol] SGT [symbol] AAGGGA | 210 |
| 3 | GKKRPVEPSPQRSPDSSTGIGKKGQQPAKKRLNF [symbol] QTGDSESVPDPQPIGEPPAGPSGL [symbol] SGT [symbol] AAGGGA | 210 |
| | ********************************************************************** | |
| | PMADNNEGADGVGSSS [symbol] NWHCDSTWLGDRVITTSTRTWALPTYNNHLYKQISNGTSGGSTNDNTYFGYST | |
| | 220 230 240 250 260 270 280 | |
| 1 | PMADNNEGADGVGSSS [symbol] NWHCDSTWLGDRVITTSTRTWALPTYNNHLYKQISNGTSGGSTNDNTYFGYST | 280 |
| 2 | PMADNNEGADGVGSSS [symbol] NWHCDSTWLGDRVITTSTRTWALPTYNNHLYKQISNGTSGGSTNDNTYFGYST | 280 |
| 3 | PMADNNEGADGVGSSS [symbol] NWHCDSTWLGDRVITTSTRTWALPTYNNHLYKQISNGTSGGSTNDNTYFGYST | 280 |
| | ********************************************************************** | |
| | PW [symbol] YFDFNRFHCHFSPRDWQRLINNNW [symbol] FRPKRLNFKLFNIQVKEVTQNE [symbol] TKTIANNLTSTIQVFTDSE | |
| | 290 300 310 320 330 340 350 | |
| 1 | PW [symbol] YFDFNRFHCHFSPRDWQRLINNNW [symbol] FRPKRLNFKLFNIQVKEVTQNE [symbol] TKTIANNLTSTIQVFTDSE | 350 |
| 2 | PW [symbol] YFDFNRFHCHFSPRDWQRLINNNW [symbol] FRPKRLNFKLFNIQVKEVTQNE [symbol] TKTIANNLTSTIQVFTDSE | 350 |
| 3 | PW [symbol] YFDFNRFHCHFSPRDWQRLINNNW [symbol] FRPKRLNFKLFNIQVKEVTQNE [symbol] TKTIANNLTSTIQVFTDSE | 350 |
| | ********************************************************************** | |
| | YQLPYVLGSAHQGCLPPFPADVFMIPQYGYLTLNNGSQAVGRSSFYCLEYFPSQMLRTGNNFEFSYNFED | |
| | 360 370 380 390 400 410 420 | |
| 1 | YQLPYVLGSAHQGCLPPFPADVFMIPQYGYLTLNNGSQAVGRSSFYCLEYFPSQMLRTGNNFEFSYNFED | 420 |
| 2 | YQLPYVLGSAHQGCLPPFPADVFMIPQYGYLTLNNGSQAVGRSSFYCLEYFPSQMLRTGNNFEFSYNFED | 420 |
| 3 | YQLPYVLGSAHQGCLPPFPADVFMIPQYGYLTLNNGSQAVGRSSFYCLEYFPSQMLRTGNNFEFSYNFED | 420 |
| | ********************************************************************** | |
| | VPFHSSYAHSQSLDRLMNPLIDQYLYYLSRTQSTGGTA [symbol] TQQLLFSQAGPNN [symbol] SAQAKNWLPGPCYRQQR | |
| | 430 440 450 460 470 480 490 | |
| 1 | VPFHSSYAHSQSLDRLMNPLIDQYLYYLSRTQSTGGTA [symbol] TQQLLFSQAGPNN [symbol] SAQAKNWLPGPCYRQQR | 490 |
| 2 | VPFHSSYAHSQSLDRLMNPLIDQYLYYLSRTQSTGGTA [symbol] TQQLLFSQAGPNN [symbol] SAQAKNWLPGPCYRQQR | 490 |
| 3 | VPFHSSYAHSQSLDRLMNPLIDQYLYYLSRTQSTGGTA [symbol] TQQLLFSQAGPNN [symbol] SAQAKNWLPGPCYRQQR | 490 |
| | ********************************************************************** | |
| | VSTTLSQNNNSNFAWTGATKYHLNGRDSLVNPGVA [symbol] ATHKDDEERFFPSSGVLMFGKQGAGKDNVDYSSV | |
| | 500 510 520 530 540 550 560 | |
| 1 | VSTTLSQNNNSNFAWTGATKYHLNGRDSLVNPGVA [symbol] ATHKDDEERFFPSSGVLMFGKQGAGKDNVDYSSV | 560 |
| 2 | VSTTLSQNNNSIFAWTGATKYHLNGRDSLVNPGVA [symbol] ATHKDDEERFFPSSGVLMFGKQGAGKDNVDYSSV | 560 |
| 3 | VSTTLSQNNNSNFAWTGATKYHLNGRDSLVNPGVA [symbol] ATHKDDEERFFPSSGVLMFGKQGAGKDNVDYSSV | 560 |
| | ********************************************************************** | |
| | [symbol] LTSEEEIKTTNPVATEQYGVVADNLQQQNAAPIVGAVNSQGALPG [symbol] VWQNRDVYLQGPIWAKIPHTDGN | |
| | 570 580 590 600 610 620 630 | |
| 1 | [symbol] LTSEEEIKTTNPVATEQYGVVADNLQQQNAAPIVGAVNSQGALPG [symbol] VWQNRDVYLQGPIWAKIPHTDGN | 630 |
| 2 | [symbol] LTSEEEIKTTNPVATEQYGVVADNLQQQNAAPIVGAVNSQGALPG [symbol] VWQNRDVYLQGPIWAKIPHTDGN | 630 |
| 3 | [symbol] LTSEEEIKTTNPVATEQYGVVADNLQQQNYIGSRGAVNSQGALPG [symbol] VWQNRDVYLQGPIWAKIPHTDGN | 630 |
| | ********************************************************************** | |
| | FHPSPLMGGFGLKHPPPQILIKNTPVPADPPTTFNQAKLASFITQYSTGQVSVEIEWELQKENSKRWNPE | |
| | 640 650 660 670 680 690 700 | |
| 1 | FHPSPLMGGFGLKHPPPQILIKNTPVPADPPTTFNQAKLASFITQYSTGQVSVEIEWELQKENSKRWNPE | 700 |
| 2 | FHPSPLMGGFGLKHPPPQILIKNTPVPADPPTTFNQAKLASFITQYSTGQVSVEIEWELQKENSKRWNPE | 700 |
| 3 | FHPSPLMGGFGLKHPPPQILIKNTPVPADPPTTFNQAKLASFITQYSTGQVSVEIEWELQKENSKRWNPE | 700 |
| | ************************************** | |
| | IQYTSNYYKSTNVDFAVNTEGTYSEPRPIGTRYLTRNL | |
| | 710 720 730 | |
| 1 | IQYTSNYYKSTNVDFAVNTEGTYSEPRPIGTRYLTRNL | 730 |
| 2 | IQYTSNYYKSTNVDFAVNTEGTYSEPRPIGTRYLTRNL | 730 |
| 3 | IQYTSNYYKSTNVDFAVNTEGTYSEPRPIGTRYLTRNL | 730 |

[symbol]

indicates data missing or illegible when filed

1. An isolated polynucleotide that is at least 85% identical to any one of SEQ ID NOs: 1-3, or an equivalent of each thereof, wherein optionally at least one or more of the nucleotides that have been modified from the wild type sequence are not modified from its corresponding wild-type sequence.

2. An isolated polynucleotide encoding the amino acid of SEQ ID NO: 4 or an amino acid at least 85% identical to the amino acid sequence of SEQ ID NO: 4, or an equivalent thereof.

3. (canceled)

4. A polynucleotide comprising the polynucleotide of SEQ ID NO: 1, or an equivalent thereof, and optionally wherein at least one or more of the nucleotides that have been modified from the wild type sequence are not modified from SEQ ID NO: 1.

5. A vector comprising the polynucleotide of claim 2.

6. The polynucleotide of claim 2 further comprising a promoter and/or an enhancer element.

7. (canceled)

8. The vector of claim 5, wherein the vector is a AAV vector.

9. The vector of claim 8, further comprising a polynucleotide encoding a capsid protein.

10. The vector of claim 9, wherein the capsid protein is a wild-type capsid protein or a mutated capsid protein.

11. An isolated cell comprising the isolated polynucleotide of claim 1.

12. A recombinant AAV vector comprising a polynucleotide encoding galactose-1-phosphate uridyl transferase (“GALT”).

13. The recombinant AAV vector of claim 12, wherein the polynucleotide encoding the GALT comprises a polynucleotide that is at least 85% identical to SEQ ID NO: 1 and optionally wherein at least one or more of the nucleotides that have been modified from the wild type polynucleotide is not modified from SEQ ID NO: 1.

14. The recombinant AAV vector of claim 12, wherein the polynucleotide encoding GALT comprises the polynucleotide set forth in SEQ ID NO: 1.

15. The recombinant AAV vector of claim 12, wherein the AAV is of the serotype AAV1, AAV2, AAV4, AAV5, AAV6, AAV7, AAV8, AAV9, AAV10, AAV11, AAV12, AAV13, AAV PHP.B, or AAV rh74.

16. The recombinant AAV vector of claim 12, wherein the AAV is selected from AAV9, rh74, a mutated rh74, or AAV PHP.B.

17.-19. (canceled)

20. The recombinant AAV vector of claim 12, further comprising a polynucleotide at least 95% identical to SEQ ID NO: 2 and optionally wherein at least one or more of the nucleotides that have been modified from the wild type polynucleotide is not modified from SEQ ID NO: 2.

21.-30. (canceled)

31. A polynucleotide encoding a modified AAVrh74 VP1 capsid protein comprising one or more modifications selected from the group of a substitution of isoleucine for asparagine at amino acid position 502, a substation of tryptophan to arginine at amino acid 505 of the VP1 of AAVrh74, and an insertion of the peptide YIG at amino acid position 591 of the VP1 of AAVrh74, or an equivalent thereof, or a polynucleotide that is at least 85% identical with the proviso that one or more modifications is identical to the amino acids in the modified AAVrh74 VP1 capsid protein.

32. A modified AAVrh74 VP1 capsid protein comprising one or more modifications selected from the group of a substitution of isoleucine for asparagine at amino acid position 502, a substation of tryptophan to arginine at amino acid 505 of the VP1 of AAVrh74, and an insertion of the peptide YIG at amino acid position 591 of the VP1 of AAVrh74, or an equivalent thereof, or a polynucleotide that is at least 85% identical with the proviso that one or more modifications are identical to the amino acids in the modified AAVrh74 VP1 capsid protein.

33.-34. (canceled)

35. A method for introducing a functional GALT enzyme into a cell, comprising contacting the cell with the polynucleotide of claim 1.

36. (canceled)

37. A method for treating galactosemia in a subject in need thereof, comprising administering to the subject a therapeutically effective amount of the polynucleotide of claim 1.

38. A method of increasing galactose metabolism in a subject suffering from galactosemia comprising administering to the subject the polynucleotide of claim 1.

39. A method of reducing a disease condition in a subject suffering from galactosemia comprising administering to the subject the polynucleotide of claim 1, wherein the disease condition selected from jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency.

40.-47. (canceled)

48. A kit comprising the polynucleotide of claim 1, and instructions for use.

49.-51. (canceled)

52. An AAV packaging system, comprising the polynucleotide of claim 1, and a helper cell line.


SEQUENCE LISTING (13 sequences)

SEQ ID NO: 1 - DNA, 1140 bp, Artificial Sequence
  Description of Artificial Sequence Synthetic polynucleotide
atgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctga

SEQ ID NO: 2 - DNA, 6237 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcggccgcagctgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagtcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgcc

SEQ ID NO: 3 - DNA, 6782 bp, Artificial Sequence
  Description of Artificial Sequence Synthetic polynucleotide
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagacagccactatgggtctaggctgcccatgtaaggaggcaaggcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcgcggccgcagctgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagtcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgaggccgcaataaaagatctttattttcattagatctgtgtgttggttttttgtgtgtctagacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 4 - protein, 379 aa, Homo sapiens
MSRSGTDPQQRQQASEADAAAATFRANDHQHIRYNPLQDEWVLVSAHRMKRPWQGQVEPQLLKTVPRHDPLNPLCPGAIRANGEVNPQYDSTFLFDNDFPALQPDAPSPGPSDHPLFQAKSARGVCKVMCFHPWSDVTLPLMSVPEIRAVVDAWASVTEELGAQYPWVQIFENKGAMMGCSNPHPHCQVWASSFLPDIAQREERSQQAYKSQHGEPLLMEYSRQELLRKERLVLTSEHWLVLVPFWATWPYQTLLLPRRHVRRLPELTPAERDDLASIMKKLLTKYDNLFETSFPYSMGWHGAPTGSEAGANWNHWQLHAHYYPPLLRSATVRKFMVGYEMLAQAQRDLTPEQAAERLRALPEVHYHLGQKDRETATIA

SEQ ID NO: 5 - protein, 736 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPVGAVNSQGALPGMVWQNRDVYLQGPIWAKPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 6 - DNA, 2215 bp, Artificial Sequence
  Description of Artificial Sequence Synthetic polynucleotide
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggagcgactcatcaacaacaactggggattccggcccaagaggctcaacttcaagctcttcaacatccaagtcaaggaggtcacgcagaatgaaggcaccaagagcatcgccaataaccttaccagcaggattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 7 - protein, 271 aa, Unknown
  Description of Unknown KAN Gene Translation sequence
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 8 - protein, 5 aa, Artificial Sequence
  Description of Artificial Sequence Synthetic peptide
YIGSR

SEQ ID NO: 9 - protein, 738 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 10 - protein, 738 aa, Artificial Sequence; identical to SEQ ID NO: 9
  Description of Artificial Sequence Synthetic polypeptide

SEQ ID NO: 11 - protein, 738 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSIFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 12 - protein, 738 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNYIGSRGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 13 - DNA, 1140 bp, Homo sapiens
atgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctga